(19) United States
(12) Patent Application Publication
(10) Pub. No.: US 2009/0170914 A1
(43) Pub. Date: Jul. 2, 2009

(54) Cermide Kinase Modulation

(76) Inventors: Frederic Bornancin, Wien (AT); Berndt Oberhauser, Wien (AT)

Correspondence Address: Charles E. Lykes, Jr., Charles E. Lykes, Jr. Attorney At Law, 501 South Fort Harrison Avenue Suite 101, Clearwater, FL, 33756-5348, US

(21) Appl. No.: 12/295,375

(22) PCT Filed: Mar. 28, 2007
(86) PCT No.: PCT/EP07/02765

§ 371 Date: Sep. 30, 2008

(30) Foreign Application Priority Data
Mar. 30, 2006 (GB) 0606429.9

(51) Int. Cl. A61K 31/423; C07D 277/82

(52) U.S. Cl. 514/367; 548/160

(57) ABSTRACT

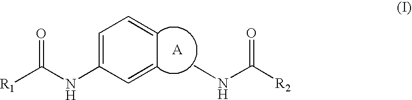

A compound of formula (I) wherein R₁ is a straight chain, branched or cyclic aliphatic, aromatic or heterocyclyl group comprising at least 8 carbon atoms, e.g. 8 to 22, R₂ is a straight chain, branched or cyclic aliphatic, aromatic or heterocyclic group comprising from 1 to 12 carbon atoms, and ring A is heterocycyl, fused with the phenyl ring to which ring A is attached comprising 5 or 6 ring members, and 1 to 4 heteroatoms selected from N, S, O; wherein certain compounds are excluded by proviso and the use of such compounds without proviso as pharmaceuticals in disorders which are mediated by ceramide kinase.

The present invention relates to modulators of ceramide kinase activity.

Sphingolipids have been considered as one of the major components of the cell membrane. Recent evidence has shown that, beyond their structural role, they can act as bioactive lipids and impact on signal transduction, in a way that is reminiscent of what is occurring with glycerophospholipids.

Physiological activity of sphingolipid metabolites include e.g. induction of apoptosis and stimulation of cell proliferation and it has been suggested that enzymes which metabolise sphingolipids are expected to participate in the induction of various diseases.

For example
ceramide which controls cell mechanisms has been suggested to be a regulator in the enzymatic reaction indicated above, e.g. it is reported that ceramide works as a second messenger of inflammatory cytokines, such as TNF-α and IL-1β, and activates arachidonic pathways, such as phospholipase A₂; and ceramide or metabolites thereof thus may be considered as an exacerbating factor in inflammatory disorders;
ceramide exacerbates the reduction of CD4⁺ T-cell accompanied by apoptosis and HIV infection of brain cells in patients infected with HIV,
it is reported that TNF-α may cause insulin resistance in type 2 diabetes mellitus as a trigger and obesity and that ceramide is involved in the downregulation of TNF-α;
it is disclosed that ceramide triggers septicemia caused by lipopolysaccharide;
it is reported that the increase of ceramide activates sphingomyelinase in the aggregating reaction of LDL which triggers atherosclerosis lesions;
it is known that ceramide promotes apoptosis of cancer cells in radiotherapy and chemotherapy;
it was shown that ceramide regulation is involved in drug resistance of leukemia cells: a decrease of ceramide level is associated with the chemoresistant condition in leukemia.

Also ceramide-1-phosphate (Cer-1-P), which is produced from ceramide by the action of ceramide kinase, e.g. by phosphorylation of the hydroxyl group at position 1 of various ceramide derivatives, e.g. including N-acylated-, such as N-hexanoyl-, N-octanoyl-, N-palmitoyl-D-erythro-sphingosine, shows physiological activities, e.g.
Cer-1-P produced by ceramide kinase upon calcium stimulation is described to regulate the release of neuronal transmitters from brain synapses, and to modulate the action of ceramide kinase is thus expected to be of value in the treatment of various neuronal disorders, e.g. including Alzheimer's disease;
Cer-1-P is believed to inhibit various normal ceramide activities, maybe through inhibition of acid sphingomyelinase and thus Cer-1-P is expected to modulate various disorders, e.g. inflammatory disorders, e.g. including chronic arthritis, HIV-infection, type 2 diabetes mellitus caused by insulin resistance as a trigger, obesity, septicemia and atherosclerosis; it is believed that such diseases may be treated by modulation of ceramide kinase;
Cer-1-P is believed to act primarily inside the cell where it facilitates vesicle transport. It has been implicated in phagocytosis, and therefore could play an important role during inflammation processes;
the mitogenic activity of exogenously added Cer-1-P has also been shown. Therefore, this sphingolipid metabolite may be relevant to cell proliferation disorders, including but not limited to cancer and psoriasis.
Cer-1-P has been reported to mediate cytokine- and calcium ionophore-induced arachidonic release, and it is further indicated that C-1-P may directly activate cytosolic PLA2; and this further evidences the possible role of Cer-1-P in inflammatory disorders;
Cer-1-P levels may also be relevant to the pathophysiology (e.g. susceptibility to retinitis pigmentosa) of the visual system.

Now surprisingly compounds which modulate, e.g. inhibit, ceramide kinase activity are provided.

In one aspect the present invention provides a compound of formula

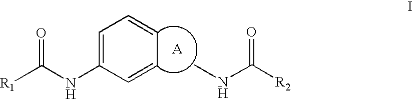

wherein
R₁ is a straight chain, branched or cyclic aliphatic, aromatic or heterocyclyl group comprising at least 8 carbon atoms, e.g. 8 to 22,
R₂ is a straight chain, branched or cyclic aliphatic, aromatic or heterocyclic group comprising from 1 to 12 carbon atoms, and
ring A is heterocycyl, fused with the phenyl ring to which ring A is attached comprising 5 or 6 ring members, preferably 5, and 1 to 4 heteroatoms selected from N, S, O; preferably two, preferably comprising at least one nitrogen atom,
with the proviso that the compounds
N-[2-(acetylamino)-6-benzothiazolyl]-tricyclo[3.3.1.13,7]decane-1-carboxamide, e.g. such as a compound of formula

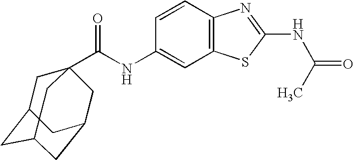

N-[2-(benzoylamino)-6-benzothiazolyl]-tricyclo[3.3.1.13,7]decane-1-carboxamide, e.g. such as a compound of formula

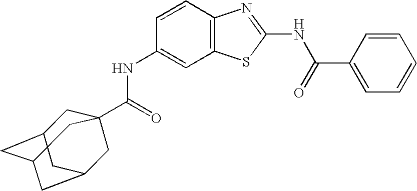

N-[2-(benzoylamino)-6-benzothiazolyl]-3-chloro-benzo[b]thiophene-2-carboxamide, e.g. such as a compound of formula

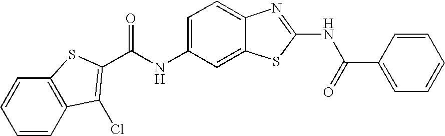

2,3-Dihydro-N-[2-[(1-oxobutyl)amino]-6-benzothiazolyl]-1,4-benzodioxin-6-carboxamide, e.g. such as a compound of formula

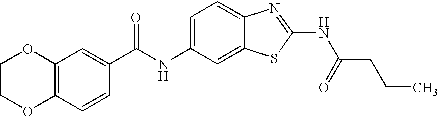

N-[2-(acetylamino)-6-benzothiazolyl]-3-chloro-benzo[b]thiophene-2-carboxamide, e.g. such as a compound of formula

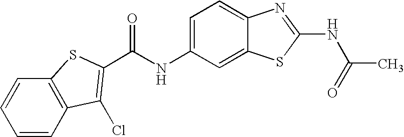

N-[2-(butyrylamino)-1,3-benzothiazol-6-yl]-3-chloro-1-benzothiophene-2-carboxamide, e.g. such as a compound of formula

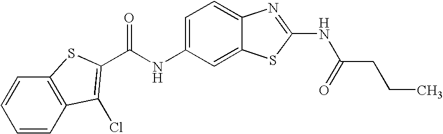

N-[2-(butyrylamino)-1,3-benzothiazol-6-yl]-1-benzofuran-2-carboxamide, e.g. such as a compound of formula

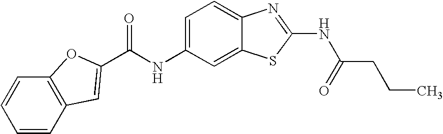

N-[2-[(cyclohexylcarbonyl)-amino]-6-benzothiazolyl]-tricyclo[3.3.1.13,7]decane-1-carboxamide,
e.g. such as a compound of formula

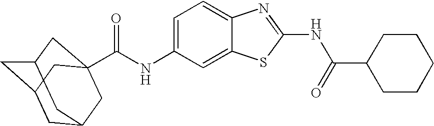

adamantane-1-carboxylic acid [2-(adamantan-1-yl)-carbonylamino-benzothiazol-6-yl]-amide, such as a compound of formula

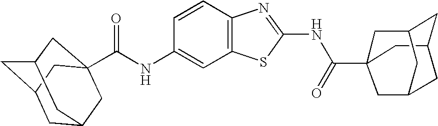

N-[2,3-dihydro-3-oxo-6-[(1-oxohexadecyl)-amino]-benzo[b]-thien-2-yl]-5-nitro-1H-indazole-1-carboxamide,
e.g. such as a compound of formula

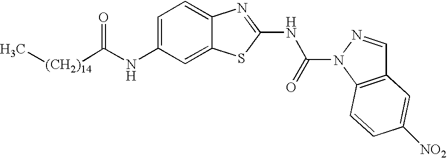

and
N-[2,3-dihydro-3-oxo-6-[(1-oxohexadecyl)-amino]-benzo[b]-thien-2-yl]-2,3,5,6-tetrafluoro-4-mercapto benzamide
e.g. such as a compound of formula

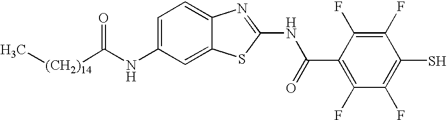

are excluded;

In a compound of formula I each single defined substitutent may be a preferred substituent, e.g. independently of each other substitutent defined

Straight chain, branched or cyclic aliphatic groups in the meaning of R₂ include e.g.
alkyl e.g. including (C₁₋₁₂)alkyl,
alkenyl e.g. including (C₂₋₁₂)alkenyl,
alkinyl e.g. including (C₂₋₁₂)alkinyl,
cycloalkyl e.g. including (C₃₋₁₂)cycloalkyl,

Aromatic groups in the meaning of R₂ e.g. include (C₆₋₁₂)aryl, such as phenyl. Heterocyclic groups in the meaning of R₂ e.g. include aromatic or aliphatic heterocyclyl, having 3 to 12 ring members, e.g. fused heterocyclyl, and 1 to 4 heteroatoms selected fro N, O, S.

Straight chain, branched or cyclic aliphatic, aromatic or heterocyclyl groups as defined herein in the meaning of R₁ may be unsubstituted or substituted, e.g. one or morefold; e.g. by groups which are conventinal substituents in organic chemistry and which groups comprise 0 to 18 carbon atoms. Such substituents in the meaning of R₁ e.g. include (C₁₋₈)alkyl, halo(C₁₋₈)alkyl, (C₂₋₈)alkenyl, (C₂₋₈)alkinyl, hydroxy, (C₁₋₈)alkoxy, halo(C₁₋₈)alkoxy, cyano, sulphur containing substituents comprising 0 to 8 carbon atoms, such as mercapto, (C₁₋₈)alkylmercapto, halogen, NO, nitro, (C₆₋₁₈)aryl, e.g. phenyl, (C₆₋₁₈)aryloxy, (C₂₋₁₂)acyl (including the carbonyl group), heterocyclyl, e.g. aliphatic or aromatic heterocyclyl, e.g. including heterocyclyl comprising fused rings (ring systems), comprising 3 to 12 ring members and 1 to 6 heteroatoms selected from N, O, S; or amino, e.g. unsubstituted amino or amino substituted by one or two (C₁₋₈)alky, (C₆₋₁₈)aryl, or by (one) (C₂₋₁₃)acyl (including the carbonyl group); wherein acyl includes (C₁₋₈)alkylcarbonyl, (C₆₋₁₈)arylcarbonyl or heterocyclylcarbonyl, e.g. aliphatic or aromatic heterocyclylcarbonyl, e.g. wherein heterocyclyl comprises single or fused rings (ring systems), comprising 3 to 18 ring members and 1 to 6 heteroatoms selected from N, O, S.

Straight chain, branched or cyclic aliphatic, aromatic or heterocyclyl groups as defined herein in the meaning of R₂ may be unsubstituted or substituted, e.g. one or morefold; e.g. by groups which are conventinal substituents in organic chemistry and which groups comprise 0 to 4 carbon atoms. Such substituents in the meaning of R₂ e.g. include (C₁₋₄)alkyl, halo(C₁₋₄)alkyl, (C₂₋₄)alkenyl, (C₂₋₄)alkinyl, (C₃₋₆)cycloalkyl, hydroxy, (C₁₋₄)alkoxy, halo(C₁₋₄)alkoxy, cyano, carboxyl and carboxylic acid derivatives, such as carboxylic acid esters or amides, comprising 1 to 4 carbon atoms (including the carbonyl group), sulphur containing substituents comprising 0 to 4 carbon atoms, e.g. including mercapto, (C₁₋₄)alkylmercapto, halogen, NO, nitro, (C₂₋₄)alkylcarbonyl (including the carbonyl group), or amino, e.g. unsubstituted amino or amino substituted by one or two (C₁₋₄)alky, or by (one) (C₂₋₄)alkylcarbonyl (including the carbonyl group).

Preferably in a compound of formula I ring A fused with the phenyl ring is a 5-membered heterocyclylic group, comprising 1 or 2 heteroatoms selected from N, O, S.

More preferably ring A together with the phenyl ring to which it is attached is a benzothiazolyl or benzoxazolyl ring, more preferably a benzothiazolyl ring,

e.g. preferably a compound of formula I is a compound of formula

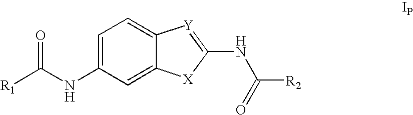

wherein

### X is S or O and Y is N, or

### Y is S or O and X is N,

preferably X is S or O and Y is N,
more preferably X is S and Y is N; and
R₁ and R₂ are as defined above.

In another aspect the present invention provides 2,6-diamido-benzothiazoles, or 2,6-diamido-benzoxazoles, wherein the carbonyl group of the aminocarbonyl group in position 6 is substituted by a straight chain, branched or cyclic aliphatic, aromatic or heterocyclyl group comprising at least 8 carbon atoms, e.g. 8 to 22, and the carbonyl group of the aminocarbonyl group in position 2 is substituted by a straight chain, branched or cyclic aliphatic, aromatic or heterocyclic group comprising from 1 to 8 carbon atoms, such as 2,6-diamido-benzothiazoles, or 2,6-diamido-benzoxazoles, e.g. 2,6-diamido-benzothiazoles, wherein the carbonyl group of the aminocarbonyl group in position 6 is substituted by a straight chain, branched or cyclic aliphatic group comprising at least 8 carbon atoms, e.g. 8 to 22, and the carbonyl group of the aminocarbonyl group in position 2 is substituted by a straight chain, branched or cyclic aliphatic or aromatic group comprising from 1 to 8 carbon atoms, with the proviso as indicated above.

Preferably in a compound of formula I

R₁ is (C₈₋₂₂)alkyl, such as (C₁₀₋₁₆)alkyl, (C₈₋₁₂)cyclohexyl, e.g. including bridged cycloalkyl, such as adamantanyl, or (C₈₋₂₂)heterocyclyl, such as fused heterocyclyl, e.g. heterocycyl fused with a phenyl ring, e.g. including benzthiazolyl, benzofuranyl, benzodioxinyl; indazoinyl, e.g. wherein alkyl, cycloalkyl or heterocyclyl is substituted or unsubstituted, e.g. unsubstituted or substituted by (C₆₋₁₈)aryl, such as phenyl. (C₁₋₄)alkyl, (C₁₋₄)alkoxy, halogen, halo(C₁₋₄)alkyl, halo (C₁₋₄)alkoxy nitro, mercapto or (C₁₋₄)alkylmercapto.

Preferably in a compound of formula I R₂ is (C₁₋₁₂)alkyl, such as (C₁₋₈)alkyl, (C₃₋₁₂)cycloalkyl (C₆₋₁₂)aryl, or heterocyclyl comprising up to 12 carbon atoms and 1 to 4 heteroatoms selected from N, O, S, including fused heterocyclyl such as benzthiazolyl, benzofuranyl, benzodioxinyl; indazoinyl,

including unsubstituted alkyl, cycloalkyl, aryl or heterocyclyl, or one or morefold substituted alkyl, cycloalkyl, aryl or heterocyclyl, e.g. unsubstituted alkyl, cycloalkyl, aryl or heterocyclyl or alkyl, cycloalkyl, aryl or heterocyclyl substituted by (C₆₋₁₈)aryl, such as phenyl. (C₁₋₄)alkyl, (C₁₋₄)alkoxy, halogen, halo(C₁₋₄)alkyl, halo (C₁₋₄)alkoxy nitro, mercapto or (C₁₋₄)alkylmercapto.

In another aspect the present invention provides a compound of formula I, which is a compound of formula

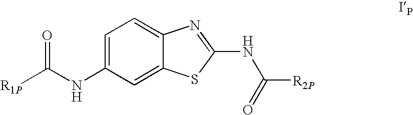

wherein
R_{1P} is (C₈₋₂₂)alkyl, or (C₈₋₁₈)cycloalkyl optionally substituted by phenyl; and
R_{2P} is (C₁₋₈)alkyl, (C₃₋₁₂)cycloalkyl, or phenyl, e.g. including (C₁₋₄)alkoxyphenyl, (C₁₋₄)dialkoxyphenyl,
with the proviso that the compounds
N-[2-(acetylamino)-6-benzothiazolyl]-tricyclo[3.3.1.13,7]decane-1-carboxamide,
N-[2-(benzoylamino)-6-benzothiazolyl]-tricyclo[3.3.1.13,7]decane-1-carboxamide, and
   adamantane-1-carboxylic acid [2-(adamantan-1-yl)-carbonylamino-benzothiazol-6-yl]-amide are excluded.

In another aspect the present invention provides a compound of formula I, selected from the group consisting of the compounds of Examples 3, 4 and 5 in TABLE 1, e.g. 3-Phenyl-adamantane-1-carboxylic acid (2-benzoylamino-benzothiazol-6-yl)-amide, N-(6-Tetradecanoylamino-benzothiazol-2-yl)-benzamide, and Adamantane-1-carboxylic acid [2-(3,4-dimethoxy-benzoylamino)-benzothiazol-6-yl]-amide.

Compounds provided by the present invention are hereinafter designated as “compound(s) of (according to) the present invention” and include a compound of formula I. A compound of formula I includes a compound of formula Iₚ and I′ₚ.

A compound of the present invention includes a compound in any form, e.g. in free form, in the form of a salt, in the form of a solvate and in the form of a salt and a solvate.

In another aspect the present invention provides a compound of the present invention in the form of a salt.

Such salts include preferably pharmaceutically acceptable salts, although pharmaceutically unacceptable salts are included, e.g. for preparation/isolation/purification purposes. A compound of the present invention in free form may be converted into a corresponding compound in the form of a salt; and vice versa. A compound of the present invention in free form or in the form of a salt and in the form of a solvate may be converted into a corresponding compound in free form or in the form of a salt in non-solvated form; and vice versa.

A compound of the present invention may exist in the form of isomers and mixtures thereof; e.g. optical isomers, diastereoisomers, cis/trans conformers. A compound of the present invention may e.g. contain asymmetric carbon atoms and may thus exist in the form of enantiomers or diastereoisomers and mixtures thereof, e.g. racemates. A compound of the present invention may be present in the (R)-, (S)- or (R,S)-configuration preferably in the (R)- or (S)-configuration regarding specified positions in the compound. E.g. if substituents attached to any of the carbonyl group of the amide groups in a compound of the present invention are alkyl or substituted cycloalkyl, a compound of the present invention may be present in the (R)-, (S)- or (R,S)-configuration preferably in the (R)- or (S)-configuration regarding any asymmetric carbon atom which may arise.

Isomeric mixtures may be separated as appropriate, e.g. according, e.g. analogously, to a method as conventional, to obtain pure isomers. The present invention includes a compound of the present invention in any isomeric form and in any isomeric mixture.

The present invention also includes tautomers of a compound of the present invention, where tautomers can exist.

In another aspect the present invention provides a process for the production of a compound of the present invention, e.g. of formula I, comprising either
a) acylating a compound of formula

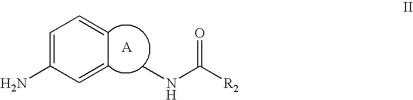

wherein R₂ is as defined above, with a compound of formula

R₁—COOH III
wherein R₁ is as defined above, e:g, and wherein the carboxylic group is in an activated form, or in the presence of an activating agent, such as (1-ethyl-3-[3-dimethylaminopropyl]carbodiimide, 1-hydroxy-7-azabenzotriazole, e.g. in the presence of a base. such as triethylamine, or
b) acylating a compound of formula

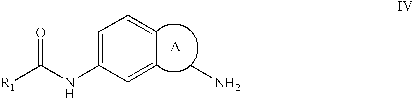

wherein R₁ is as defined above, with a compound of formula

R_{2″}—COOH V
wherein R₂ is as defined above, e:g, and wherein the carboxylic group is in an activated form, or in the presence of an activating agent, such as (1-ethyl-3-[3-dimethylaminopropyl]carbodiimide, 1-hydroxy-7-azabenzotriazole, e.g. in the presence of a base, such as triethylamine, and
   isolating a compound of formula I obtained from the reaction mixture:

2,6-diamido-benzothiazoles, or 2,6-diamido-benzoxazoles, wherein the nitrogen of the amino group in position 6 is substituted by a straight chain, branched or cyclic aliphatic, aromatic or heterocyclyl group as defined above, and wherein the nitrogen of the amino group in position 2 is substituted by a straight chain, branched or cyclic aliphatic, aromatic or heterocyclic group as defined above, may be e.g. provided by either
a) acylating a 6-amino-2-amido-benzothiazole, or a 6-amino-2-amido-benzoxazole, respectively, wherein the carbonyl carbon atom of the amide group in position 2 is substituted by a straight chain, branched or cyclic aliphatic, aromatic or heterocyclic group comprising from 1 to 12 carbon atoms, with a straight chain, branched or cyclic aliphatic, aromatic or heterocyclic carboxylic acid comprising at least 8, e.g. 8 to 22, carbon atoms beside the carbonyl carbon atom, e:g. wherein the carboxylic acid is in an activated form, or in the presence of an activating agent, e.g. (1-ethyl-3-[3-dimethylaminopropyl]carbodiimide, 1-hydroxy-7-azabenzotriazole, e.g. in the presence of a base. such as triethylamine, or
b) acylating a 2-amino-6-amido-benzothiazole, or a 2-amino-6-amido-benzoxazole, respectively, wherein the carbon atom of the carbonyl group in the amide group in position 6 is substituted by a straight chain, branched or cyclic aliphatic, aromatic or heterocyclyl group comprising at least 8 carbon atoms, e.g. 8 to 22, with a straight chain, branched or cyclic aliphatic, aromatic or heterocyclic carboxylic acid comprising from 1 to 12 carbon atoms beside the carbonyl carbon atom, e:g. wherein the carboxylic acid is in an activated form, or in the presence of an activating agent, e.g. (1-ethyl-3-[3-dimethylaminopropyl]carbodiimide, 1-hydroxy-7-azabenzotriazole, e.g. in the presence of a base. such as triethylamine, and
   isolating 2,6-diamido-benzothiazoles, or 2,6-diamido-benzoxazoles, wherein the carbon atom of the carbonyl group of the amido group in position 6 is substituted by a straight chain, branched or cyclic aliphatic, aromatic or heterocyclyl group as defined above, and wherein the carbon atom of the carbonyl group in the amido group in position in position 2 is substituted by a straight chain, branched or cyclic aliphatic, aromatic or heterocyclic group as defined above.

In an intermediate of formula II, III, IV or V (starting materials), functional groups, if present, optionally may be in protected form or in the form of a salt, if a salt-forming group is present. Protecting groups, optionally present, may be removed at an appropriate stage, e.g. according, e.g. analogously, to a method as conventional

A compound of formula I thus obtained may be converted into another compound of formula I, e.g. or a compound of formula I obtained in free form may be converted into a salt of a compound of formula I and vice versa.

The above reaction is an acylation reaction or a peptidic bond forming reaction and may be carried out as appropriate, e.g. according, e.g. analogously, to a conventional acylation or peptidic bond forming reaction.

Intermediates (starting materials) of formula II, III, IV or V are known or may be prepared according, e.g. analogously, to a method as conventional or as described herein. For example, a compound of formula II may be e.g. prepared by reducing a compound of formula

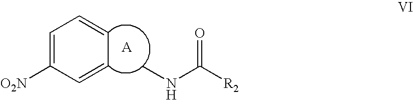

wherein R₂ is as defined above, in the presence of Sn and HCl, and isolating a compound of formula II obtained from the reaction mixture.

A compound of formula VI may be e.g. obtained by acylating a compound of formula

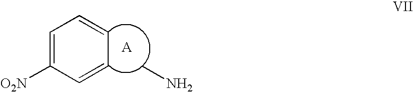

with a compound of formula

R₂—COOH V

wherein R₂ is as defined above, e.g. and wherein the carboxylic group is in an activated form, such as in the form of an carboxylic acid halogenide, and isolating a compound of formula VI from the reaction mixture.

A compound of formula IV may be e.g. obtained by acylating a compound of formula

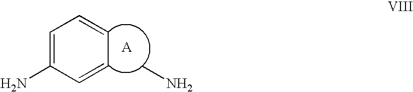

with a compound of formula

R₁—COOH III

wherein R₁ is as defined above, e:g, and wherein the carboxylic group is in an activated form, or in the presence of an activating agent, e.g. (1-ethyl-3-[3-dimethylaminopropyl]carbodiimide, 1-hydroxy-7-azabenzotriazole, e.g. in the presence of a base, such as triethylamine, and isolating a compound of formula IV obtained from the reaction mixture.

A compound of formula VIII may be e.g. obtained by reducing a compound of formula VII with hydrogen, e.g. in the presence of Raney-Ni.

In another aspect the present invention provides a compound of formula IV, wherein ring A and R₁ are as defined above, e.g. a compound of formula IV, wherein R₁ is R₁ₚ; e.g. a compound of formula IV, wherein ring A is a benzothiazolyl or benzoxazolyl group, such as a benzothiazolyl group, e.g. a compound of formula

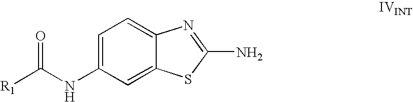

wherein R₁ is as defined above, such as a compound of formula iV_{INT}, wherein R₁ is adamantanyl, phenyladamantanyl or dodecancyl, e.g. n-dodecanyl. such as a compound of formula iV_{INT}, wherein R₁ is as defined in TABLE 1 in the Example part.

Any compound described herein, e.g. a compound of the present invention and intermediates of formula II, III, IV, V, VI, VII and VIII may be prepared as appropriate, e.g. according, e.g. analogously, to a method as conventional, e.g. or as specified herein.

The compounds of the present invention, e.g. including a compound of formula I, Iₚ and I′ₚ, exhibit pharmacological activity and are therefore useful as pharmaceuticals. E.g., the compounds of the present invention are found to inhibit ceramide kinase activity. Such inhibition may be e.g. shown in the In vitro Ceramide Kinase Assay and in the Cell-based Ceramide Kinase Assay as described below; e.g. and additionally in the Ceramide Kinase Assay as described in Christine Graf, Philipp Rovina, Loic Tauzin, Andrea Schanzer and Frederic Bornancin, “Enhanced ceramide-induced apoptosis in ceramide kinase overespressing cells”, Biochemical and Biophysical Communications 354 (2007), p. 309-314. Ceramide kinase overexpressing cells exhibit enhanced sensitivity to ceramide-mediated apoptosis. This is a direct consequence of ceramide kinase activity. Thus, inhibitors of ceramide kinase are capable of reverting this enhanced sensitivity back to the usual level found in parental cells (i.e. not overexpressing ceramide kinase). Accordingly, compounds inactive towards ceramide kinase have no effect in this assay

### Abbreviations Used in Both Following Assay Descriptions

DETAPAC diethylenetriaminepentaacetic acid
DMEM medium Dulbecco's modified Eagie's medium
DTT dithiothreitol
EGTA Ethylene glycol-bis(beta-aminoethyl ether)-N,N,N′,N′-tetraacetic acid
MOPS 3-(N-morpholino)propane sulfonic acid
NBD 7-nitro-2,1,3-benzoxadiazol-4-yl
HBBS Hank&'s BSS (Balanced salt solution, phosphate-buffered to pH 7.0-7.2)

### In Vitro Ceramide Kinase Assay

That assay is performed on recombinant GST-His-CerK produced in insect cells and purified to 90% using single step chromatography on Nickel-agarose. The purified protein is frozen in aliquots containing 0.5 mg/ml GST-His-CerK in 10 mM MOPS, pH7.2, 300 mM KCl, 500 mM imidazole, 2.5 mM DTT, 5% Glycerol, 0.01% Triton X-100.

CerK activity assays are performed in total volumes of 100 μl with the following components (final concentrations): 180 μM N-octanoyl-sphingosine (C8-ceramide), 1 mM cardiolipin, 1.5% β-octylglucoside, 0.2 mM DETAPAC, 20 mM MOPS, pH 7.2, 50 mM NaCl, 1 mM DTT, 2 mM EGTA, 3 mM CaCl₂, 500 μM (γ-³²P)ATP (40-100 mCi/mmol). Reactions are started by addition of protein samples (20 μl/assay). The final GST-His-CerK protein concentration in the assays was 40 ng/μl. Compounds stock solutions were prepared in DMSO at 10 mM and diluted in assay mixes (final DMSO concentration was 1%). Incubations are carried out for 15 min at 30° C. Reactions are stopped by adding 1 ml of chloroform/methanol 1:1 and 430 μl M KCl in 20 mM MOPS pH 7.2. 400 μl, of the organic phase are removed, further extracted with 300 μl M KCl in 20 mM MOPS pH 7.2. After vortexing followed by short centrifugation, 200 μl, of the organic phase are removed and counted directly. Ceramide-1-phosphate is the only phosphorylated product detectable in the final organic phase under these conditions.

### Cell-Based Ceramide Kinase Assay

Control COS-1 cells in 24-well plates or COS-1 cells stably overexpressing ceramide kinase, are treated with fluorescent NBD labeled C6-ceramide for 3 hr in 10% serum-containing DMEM medium. Subsequently, cells are washed with 500 μl, of HBSS supplemented with 10 mM of EDTA. Lipids are extracted with 100 μl, of CH₃OH. After transfer in Eppendorf tubes, 100 μl, of CHCl₃ are added as well as 150 μl of HBSS/EDTA. After vortexing and short centrifugation, the organic phase is collected and dried out using a speed-vac. The dried lipids are finally dissolved into CHCl₃/CH₃OH and processed with thin layer chromatography analysis using butanol/acetic acid/water 3:1:1 as the eluent. Compounds prepared at 10 mM in DMSO are diluted directly into the cell culture medium to reach the appropriate concentrations. DMSO is used as a vehicle control.

The EC₅₀ in the above described assays is determined by use of different concentration ranges of the compounds tested. The activity obtained without compound is set at 100%.

In the assays described above the compounds of the present invention inhibit purified and intracellular ceramide kinase activity, e.g. the compounds of the present invention inhibit binding of ceramide to ceramide kinase. In the assays described above the compounds of the present invention show EC₅₀ values from the low nanomolar range up to the low micromolar range.

Furthermore the compounds of the present invention are active in the Ceramide Kinase Assay as described in Christine Graf et al, supra.

The compounds of the present invention are ceramide kinase (CERK) antagonists and are useful for the treatment of disorders mediated by CERK activity.

Disorders as used herein include diseases.

Disorders mediated by CERK activity which are prone to be successfully treated with CERK antagonists, e.g. with compounds of the present invention, include disorders, wherein the activity of CERK play a causal or contributory role, such as such as immune responses initiated by dendritic cells (DCs), monocytes or lymphocytes.

Such disorders (diseases) include but are not limited to

e.g. include
disorders associated with inflammation
e.g. including (chronic) inflammatory disorders, disorders related with the inflammation of the bronchi, e.g. including bronchitis, cervix, e.g. including cervicitis, conjunctiva, e.g. conjunctivitis, esophagus, e.g. esophagitis, heart muscle, e.g. myocarditis, rectum, e.g. proctitis, sclera, e.g. scleritis, gums, involving bone, pulmonary inflammation (alveolitis), airways, e.g. asthma, such as bronchial asthma, acute respiratory distress syndrome (ARDS), inflammatory skin disorders such as contact hypersensitivity, atopic dermatitis; fibrotic disease (e.g., pulmonary fibrosis), encephilitis, inflammatory osteolysis,
disorders associated with conditions of the immune system,
such as autoimmune disorders e.g. including Graves' disease, Hashimoto's disease (chronic thyroiditis), multiple sclerosis, rheumatoid arthritis, arthritis, gout, osteoarthritis, scleroderma, lupus syndromes, systemic lupus erytomatosis, Sjoegren's syndrome, psoriasis, inflammatory bowel disease, including Crohn's disease, colitis, e.g. ulcerative colitis; sepsis, septic shock, autoimmune hemolytic anemia (AHA), autoantibody triggered urticaria, pemphigus, nephritis, glomerulonephritis, Goodpastur syndrom, ankylosing spondylitis, Reiter's syndrome, polymyositis, dermatomyositis, cytokine-mediated toxicity, interleukin-2 toxicity, alopecia greata, uveitis, lichen planus, bullous pemphigoid, myasthenia gravis, type I diabetes mellitus, immune-mediated infertility such as premature ovarian failure, polyglandular failure, hypothyroidism, pemphigus vulgaris, pemphigus 1-oliaceus, paraneoplastic pemphigus, autoimnune hepatitis including that associated with hepatitis B virus (HBV) and hepatitis C virus (HCV), Addison's disease, autoimmune skin diseases, such as psoriasis, dermatitis herpetiformis, epidermolysis bullosa, linear IgA bullous dermatosis, epidermolysis bullosa acquisita, chronic bullous disease of childhood, pernicious anemia, hemolytic anemia, vitiligo, type I, type II and type III autoimmune polyglandular syndromes, Autoimmune Hypoparathyroidism, Autoimmune Hypophysitis, Autoimmune Oophoritis, Autoimmune Orchitis, pemphigoid gestationis, cicatricial pemphigoid, mixed essential cryoglobulinemia, immune thrombocytopenic purpura, Goodpasture's syndrome, autoimmune neutropenia, Eaton-Lambert myasthenic syndrome, stiff-man syndrome, encephalomyelitis, acute disseminated encephalomyelitis, Guillain-Barre syndrome, cerebellar degeneration, retinopathy, primary biliary sclerosis, sclerosing cholangitis autoimmune hepatitis, gluten-sensitive enteropathy, reactive arthritides, polymyositis/dermatomyositis, mixed connective tissue disease, Bechet's syndrome, polyarteritis nodosa allergic anguitis and granulomatosis (Churg-Strauss disease), polyangiitis overlap syndrome (hypersensitivity) vasculitis, Wegener's granulomatosis, temporal arteritis Kawasaki's disease, sarcoidosis, cryopathies, Celiac disease,
disorders associated with transplantation,
e.g. including transplant rejection crisis and other disorders following transplantation, such as organ or tissue (xeno)transplant rejection, e.g. for the treatment of recipients of e.g. heart, lung, combined heart-lung, liver, kidney, pancreatic, skin, corneal transplants, graft versus host disease, such as following bone marrow transplantation, ischemic reperfusion injury,
disorders associated with cytokine-mediated toxicity,
e.g. including interleukin-2 toxicity,
disorders associated with the bone,
e.g. including osteoporosis, osteoarthritis,
disorders associated with rheumatic disorders,
e.g. including arthritis, rheumatoid arthritis, osteoarthritis, psoriatic arthritis, crystal arthropathies, gout, pseudogout, calcium pyrophosphate deposition disease, lupus syndromes, systemic lupus erythematosus, sclerosis, sclerodema, multiple sclerosis, artherosclerosis, arteriosclerosis, spondyloarthropathies, systemic sclerosis, reactive arthritis, Reiter's syndrome, ankylosing spondylitis, polymyositis,
disorders associated with sarcoidosis,
disorders associated with pain,
e.g. associated with CNS disorders, such as multiple sclerosis, spinal cord injury, sciatica, failed back surgery syndrome, traumatic brain injury, epilepsy, Parkinson's disease, post-stroke, and vascular lesions in the brain and spinal cord (e.g., infarct, hemorrhage, vascular malformation);
non-central neuropathic pain, e.g. including that associated with post mastectomy pain, phantom feeling, reflex sympathetic dystrophy (RSD), trigeminal neuralgiaradioculopathy, post-surgical pain, HIV/AIDS related pain, cancer pain, metabolic neuropathies (e.g., diabetic neuropathy, vasculitic neuropathy secondary to connective tissue disease), paraneoplastic polyneuropathy associated, for example, with carcinoma of lung, or leukemia, or lymphoma, or carcinoma of prostate, colon or stomach, trigeminal neuralgia, cranial neuralgias, and post-herpetic neuralgia;
pain associated with peripheral nerve damage, central pain (i.e. due to cerebral ischemia) and various chronic pain i.e., lumbago, back pain (low back pain), inflammatory and/or rheumatic pain;
headache pain (for example, migraine with aura, migraine without aura, and other migraine disorders), episodic and chronic tension-type headache, tension-type like headache, cluster headache, and chronic paroxysmal hemicrania;
visceral pain such as pancreatits, intestinal cystitis, dysmenorrhea, irritable Bowel syndrome, Crohn's disease, biliary colic, ureteral colic, myocardial infarction and pain syndromes of the pelvic cavity, e.g., vulvodynia, orchialgia, urethral syndrome 15 and protatodynia;
acute pain, for example postoperative pain, and pain after trauma;
disorders associated with infectious disorders, e.g. with chronic infectous conditions, e.g. including bacterial disorders, otitis media, Lyme disease, thryoditis, viral disorders, parasitic disorders, fungal disorders, malaria, e.g. malaria anemia, sepsis, severe sepsis, septic shock, e.g. endotoxin-induced septic shock, exotoxin-induced toxic shock, infective (true septic) shock, septic shock caused by Gram-negative bacteria, pelvic inflammatory disease, AIDS, enteritis, pneumonia; meningitis, encephalitis,
disorders associated with myasthenia gravis,
disorders associated with nephritis,
e.g. including glomerulonephritis, interstitial nephritis, Wegener's granulomatosis, fibrosis,
disorders associated with cancer and cell overproliferation,
e.g. including premalignant conditions, hyperproliferative disorders, all type of cancers, cancers whether primary or metastatic, cervical and metastatic cancer, cancer originating from uncontrolled cellular proliferation, solid tumors, unresponsiveness to normal death-inducing signals (immortalization), increased cellular motility and invasiveness, increased ability to recruit blood supply through induction of new blood vessel formation (angiogenesis, e.g. including unsufficient ability to recruit blood supply, disorders characterised by odified angiogenesis, tumor associated angiogenesis), genetic instability, dysregulated gene expression, solid tumors, such as described in WO02066019, including nonsmall cell lung cancer, cervical cancer; tumor growth, lymphoma, B-cell or T-cell lymphoma, benign tumors, benign dysproliferative disorders, renal carcinoma, esophageal cancer, stomach cancer, renal carcinoma, bladder cancer, breast cancer, colon cancer, lung cancer, melanoma, nasopharyngeal cancer, osteocarcinoma, ovarian cancer, uterine cancer; prostate cancer, skin cancer, leukemia, tumor neovascularization, angiomas, myelodysplastic disorders, unresponsiveness to normal death-inducing signals (immortalization), increased cellular motility and invasiveness, genetic instability, dysregulated gene expression, (neuro)endocrine cancer (carcinoids), blood cancer, lymphocytic leukemias, neuroblastoma; soft tissue cancer, cancer prevention, e.g. prevention of metastasis,
disorders associated with diabetic conditions,
e.g. including diabetes (type I diabetes, type II diabetes, gestational diabetes), diabetic retinopathy, insulin-dependent diabetes, diabetes mellitus, gestational diabetes), insulin hyposecretion, obesity;
disorders associated with endiometriosis, testicular dysfunctions,
disorders associated with the brain and the nerves,
neurodegenerative disorders, e.g. including disorders of the central nervous system as well as disorders of the peripheral nervous system, e.g. CNS disorders including central nervous infections, brain injuries, cerebrovascular disorders and their consequences, Parkinson's disease, corticobasal degeneration, motor neuron disease, dementia including ALS, multiple sclerosis, traumatic disorders, including trauma and inflammatory consequences of trauma, traumatic brain injury, stroke, post-stroke, post-traumatic brain injury,
small-vessel cerebrovascular disease, eating disorders; further dementias, e.g. including Alzheimer's disease, vascular dementia, dementia with Lewy-bodies, frontotemporal dementia and Parkinsonism linked to chromosome 17, frontotemporal dementias, including Pick's disease, progressive nuclear palsy, corticobasal degeneration, Huntington's disease, thalamic degeneration, Creutzfeld Jakob dementia, HIV dementia, schizophrenia with dementia, Korsakoff's psychosis,
cognitive-related disorders, such as mild cognitive impairment, age-associated memory impairment, age-related cognitive decline, vascular cognitive impairment, attention deficit disorders, attention deficit hyperactivity disorders, and memory disturbances in children with learning disabilities; conditions associated with the hypothalamic-pituitary-adrenal axis,
neuronal disorders, e,g, including neuronal migration disorders, hypotonia (reduced muscle tone), muscle weakness, seizures, developmental delay (physical or mental development difficulty), mental retardation, growth failure, feeding difficulties, lymphedema, microcephaly, symptoms affecting the head and the brain, motor dysfunction;
disorders associated with the eye,
e.g. including uveoritinitis, vitreoretinopathy, corneal disease, iritis, iridocyclitis, cateracts, uveitis, diabetic retinopathy, retinitis pigmentosa, conjunctivits, keratitis,
disorders associated with the gastrointestinal tract
e.g. including colitis, inflammatory bowel disease, Crohn's disease, ulcerative colitis, peptic ulceration, gastritis, oseophagitis,
disorders associated with the heart and vascular conditions
e.g. including cardiovascular disorders, e.g. including cardiac failure, cardiac infarction, cardiac hypertrophy, heart failure, e.g. including all forms of heart pumping failures such as high-output and low-output, acute and chronic, right sided or left-sided, systolic or diastolic, independent of the underlying cause; myocardial infarction (MI), MI prophylaxis (primary and secondary prevention), acute treatment of Ml, prevention of complications; heart disorders, proliferative vascular disorders, vasculitides, polyarteritis nodosa, inflammatory consequences of ischemia, ischemic heart disease, myocardial infarction, stroke, peripheral vascular disease, pulmonary hypertension,
ischemic disorders, e.g. including myocardial ischemia, e.g. stable angina, unstable angina, angina pectoris, bronchitis; asymptomatic arrhythmias such as all forms of atrial and ventricular tachyarrhythmias, atrial tachycardia, atrial flutter, atrial fibrillation, atrioventricular reentrant tachycardia, preexitation syndrome, ventricular tachycardia, ventricular flutter, ventricular fibrillation, bradycardic forms of arrhythmias; arrhythmia, chronic obstructive pulmonary disease,
hypertension, such as systolic or diastolic high blood pressure, e.g essetnial and secondary hypertension, e.g. including hypertensive vascular disorders, such as primary as well as all kinds of secondary arterial hypertension, renal, endocrine, neurogenic and others;
peripheral vascular disorders in which arterial and/or venous flow is reduced resulting in an imbalance between blood supply and tissue oxygen demand, e.g. including artherosclerosis, chronic peripheral arterial occlusive disease (PAOD), acute arterial thrombosis and embolism, inflammatory vascular disorders, Raynaud's phenomenon and venous disorders; atherosclerosis, a disease in which the vessel wall is remodeled, e.g. including accumulation of cells, both smooth muscle cells and monocyte/macrophage inflammatory cells, in the intima of the vessel wall;
hypotension,
disorders associated with the liver and the kidneys,
e.g. including renal disorders, kidney disorders, e.g. acute kidney failure, acute renal disease, liver disorders, e.g. cirrhosis, hepatitis, liver failure, cholestasis, acute/chronic hepatitis, sclerosing cholangitis, primary billiary cirrhosis, acute/chronic interstitial/glomerulonephritis, granulomatous diseases,
disorders associated with stomach or pancreas conditions
e.g. including stomach disorders, e.g. gastric ulcer, gastrointestinal ulcer, pancreatic disorders, pancreatic fatigue,
disorders associated with the respiratory tract and lung
e.g. including pulmonary disorders, chronic pulmonary disease, acute (adult) respiratory distress syndrome (ARDS), asthma, asthma bronchitis, bronchiectasis, diffuse interstitial lung disorders, pneumoconioses, fibrosing aveolitis, lung fibrosis,
disorders associated with skin and connective tissue conditions
e.g. including eczema, atopic dermatitis, contact dermatitis, psoriasis, acne, dermatomyositis, Sjörgen's syndrome, Churg-Struass syndrome, sunburn, skin cancer, wound healing, urticaria, toxic epidermal necrolysis,
disorders associated with allergic conditions,
e.g. including delayed-type hypersensitivity, allergic conjunctivitis, drug allergies, rhinitis, allergic rhinitis, vasculitis, contact dermatitis.

Disorders, e.g. including diseases, mediated by CERK activity which are prone to be successfully treated with CERK agonists, such as compounds of the present invention, preferably include inflammation, immune, e.g. autoimmune and allergic disorders, such as rheumatoid arthritis, inflammatory bowel disease, systemic lupus erytomatosis, multiple sclerosis, transplant rejection crisis, disorders associated with skin and connective tissue conditions such as psoriasis, cancer and AIDS, more preferably rheumatoid arthritis, inflammatory bowel disease, systemic lupus erytomatosis, multiple sclerosis, psoriasis.

Treatment includes treatment and prophylaxis (prevention).

For such treatment, the appropriate dosage will, of course, vary depending upon, for example, the chemical nature and the pharmakokinetic data of a compound of the present invention used, the individual host, the mode of administration and the nature and severity of the conditions being treated. However, in general, for satisfactory results in larger mammals, for example humans, an indicated daily dosage includes a range
from about 0.001 g to about 1.5 g, such as 0.001 g to 1.5 g;
from about 0.01 mg/kg body weight to about 20 mg/kg body weight, such as 0.01 mg/kg body weight to 20 mg/kg body weight,

for example administered in divided doses up to four times a day.

A compound of the present invention may be administered to larger mammals, for example humans, by similar modes of administration at similar dosages than conventionally used with other mediators, e.g. low molecular weight inhibitors, of CERK activity.

A compound of the present invention may be administered by any conventional route, for example enterally, e.g. including nasal, buccal, rectal, oral, administration; parenterally, e.g. including intravenous, intramuscular, subcutanous administration; or topically; e.g. including epicutaneous, intranasal, intratracheal administration; via medical devices for local delivery,

e.g. stents,
e.g. in form of coated or uncoated tablets, capsules, (injectable) solutions, solid solutions, suspensions, dispersions, solid dispersions; e.g. in the form of ampoules, vials, in the form of creams, gels, pastes, inhaler powder, foams, tinctures, lip sticks, drops, sprays, or in the form of suppositories.

The compounds of the present invention may be administered in the form of a pharmaceutically acceptable salt, or in free form; optionally in the form of a solvate. A compound of the present invention in the form of a salt and/or in the form of a solvate exhibit the same order of activity as a compound of the present invention in free form.

In another aspect the present invention provides
a compound of the present invention for use as a pharmaceutical,
the use of a compound of the present invention as a pharmaceutical,
e.g. for the treatment of disorders mediated by ceramide kinase activity.

In a preferred embodiment the present invention provides the use of compounds as set out in TABLE 1 in the example part herein as pharmaceuticals.

For pharmaceutical use one or more compounds of the present invention may be used, e.g. one, or a combination of two or more compounds of the present invention, preferably one compound of the present invention is used.

A compound of the present invention may be used as a pharmaceutical in the form of a pharmaceutical composition.

In another aspect the present invention provides a pharmaceutical composition comprising a compound of the present invention in association with at least one pharmaceutically acceptable excipient, e.g. appropriate carrier and/or diluent, e.g. including fillers, binders, disintegrants, flow conditioners, lubricants, sugars or sweeteners, fragrances, preservatives, stabilizers, wetting agents and/or emulsifiers, solubilizers, salts for regulating osmotic pressure and/or buffers.

In another aspect the present invention provides
a pharmaceutical composition of the present invention for use of treating disorders which are mediated by ceramide kinase activity.
the use of a pharmaceutical composition of the present invention for treating disorders which are mediated by ceramide kinase activity.

In a further aspect the present invention provides a method of treating disorders which are mediated by ceramide kinase activity, e.g. including disorders as specified above, which treatment comprises administering to a subject in need of such treatment a therapeutically effective amount of a compound of the present invention; e.g. in the form of a pharmaceutical composition.

In another aspect the present invention provides
a compound of the present invention for the manufacture of a medicament,
the use of a compound of the present invention for the manufacture of a medicament, e.g. in the form of a pharmaceutical composition,
   for the treatment of disorders which are mediated by ceramide kinase activity.

A compound of the present invention may be used for any method or use as described herein alone or in combination with one or more, at least one, other, second drug substance.

In another aspect the present invention provides
A combination of a compound of the present invention with at least one second drug substance;
A pharmaceutical combination comprising a compound of the present invention in combination with at least one second drug substance;
A pharmaceutical composition comprising a compound of the present invention in combination with at least one second drug substance and one or more pharmaceutically acceptable excipient(s);
A compound of the present invention in combination with at least one second drug substance, e.g. in the form of a pharmaceutical combination or composition, for use in any method as defined herein, e.g.
   A combination, a pharmaceutical combination or a pharmaceutical composition, comprising a compound of the present invention and at least one second drug substance for use as a pharmaceutical;
   The use as a pharmaceutical of a compound of the present invention in combination with at least one second drug substance, e.g. in the form of a pharmaceutical combination or composition;
A method for treating disorders mediated by CERK activity in a subject in need thereof, comprising co-administering, concomitantly or in sequence, a therapeutically effective amount of a compound of the present invention and at least one second drug substance, e.g. in the form of a pharmaceutical combination or composition;
A compound of the present invention in combination with at least one second drug substance, e.g. in the form of a pharmaceutical combination or composition, for use in the preparation of a medicament for use in disorders mediated by CERK activity.

Combinations include fixed combinations, in which a compound of the present invention and at least one second drug substance are in the same formulation; kits, in which a compound of the present invention and at least one second drug substance in separate formulations are provided in the same package, e.g. with instruction for co-administration; and free combinations in which a compound of the present invention and at least one second drug substance are packaged separately, but instruction for concomitant or sequential administration are given.

In another aspect the present invention provides
A pharmaceutical package comprising a first drug substance which is a compound of the present invention and at least one second drug substance, beside instructions for combined administration;
A pharmaceutical package comprising a compound of the present invention beside instructions for combined administration with at least one second drug substance;
A pharmaceutical package comprising at least one second drug substance beside instructions for combined administration with a compound of the present invention.

Treatment with combinations according to the present invention may provide improvements compared with single treatment.

In another aspect the present invention provides
A pharmaceutical combination comprising an amount of a compound of the present invention and an amount of a second drug substance, wherein the amounts are appropriate to produce a synergistic therapeutic effect;
A method for improving the therapeutic utility of a compound of the present invention comprising co-administering, e.g. concomitantly or in sequence, of a therapeutically effective amount of a compound of the present invention and a second drug substance.
A method for improving the therapeutic utility of a second drug substance comprising co-administering, e.g. concomitantly or in sequence, of a therapeutically effective amount of a compound of the present invention and a second drug substance.

A combination of the present invention and a second drug substance as a combination partner may be administered by any conventional route, for example as set out above for a compound of the present invention. A second drug may be administered in dosages as appropriate, e.g. in dosage ranges which are similar to those used for single treatment, or, e.g. in case of synergy, even below conventional dosage ranges.

Pharmaceutical compositions according to the present invention may be manufactured according, e.g. analogously, to a method as conventional, e.g. by mixing, granulating, coating, dissolving or lyophilizing processes. Unit dosage forms may contain, for example, from about 0.1 mg to about 1500 mg, such as 1 mg to about 1000 mg.

Pharmaceutical compositions comprising a combination of the present invention and pharmaceutical compositions comprising a second drug as described herein, may be provided as appropriate, e.g. according, e.g. analogously, to a method as conventional, or as described herein for a pharmaceutical composition of the present invention.

By the term “second drug substance” is meant a chemotherapeutic drug, especially any chemotherapeutic agent other than a compound of the present invention, such as a compound of formula I.

For example, a second drug substance as used herein include

anti-inflammatory drugs, immunomodulatory drugs, anticancer drugs, antiviral drugs, antiallergic drugs, anaesthetic drugs.

Anti-inflammatory and/or immunomodulatory drugs, e.g. including antiviral drugs, which are prone to be useful in combination with a compound of the present invention include e.g.
mediators, e.g. inhibitors, of mTOR activity, including rapamycin of formula

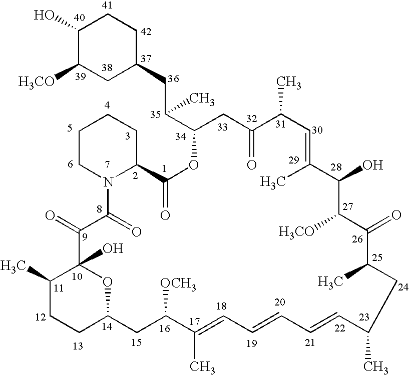

and rapamycin derivatives, e.g. including
40-O-alkyl-rapamycin derivatives, such as 40-β-hydroxyalkyl-rapamycin derivatives, such as 40-O-(2-hydroxy)-ethyl-rapamycin (everolimus),
32-deoxo-rapamycin derivatives and 32-hydroxy-rapamycin derivatives, such as 32-deoxorapamycin,
16-O-substituted rapamycin derivatives such as 16-pent-2-ynyloxy-32-deoxorapamycin,
16-pent-2-ynyloxy-32 (S or R)-dihydro-rapamycin, 16-pent-2-ynyloxy-32(S or R)-dihydro-40-O-(2-hydroxyethyl)-rapamycin,
rapamycin derivatives which are acylated at the oxygen group in position 40, e.g. 40-[3-hydroxy-2-(hydroxy-methyl)-2-methylpropanoate]-rapamycin (also known as CC1779),
rapamycin derivatives which are substituted in 40 position by heterocyclyl, e.g. 40-epi-(tetrazolyl)-rapamycin (also known as ABT578),
the so-called rapalogs, e.g. as disclosed in WO9802441, WO0114387 and WO0364383, such as AP23573, and
compounds disclosed under the name TAFA-93, AP23464, AP23675, AP23841 and biolimus (e.g. biolimus A9).
mediators, e.g. inhibitors, of calcineurin, e.g. cyclosporin A, FK 506;
ascomycins having immuno-suppressive properties, e.g. ABT-281, ASM981;
corticosteroids; cyclophosphamide; azathioprene; leflunomide; mizoribine;
mycophenolic acid or salt; e.g. sodium, mycophenolate mofetil;
15-deoxyspergualine or an immunosuppressive homologue, analogue or derivative thereof;
mediators, e.g. inhibitors, of bcr-abl tyrosine kinase activity;
mediators, e.g. inhibitors, of c-kit receptor tyrosine kinase activity;
mediators, e.g. inhibitors, of PDGF receptor tyrosine kinase activity, e.g. Gleevec (imatinib);
mediators, e.g. inhibitors, of p38 MAP kinase activity,
mediators, e.g. inhibitors, of VEGF receptor tyrosine kinase activity,
mediators, e.g. inhibitors, of PKC activity, e.g. as disclosed in WO0238561 or WO0382859, e.g. the compound of Example 56 or 70;
mediators, e.g. inhibitors, of JAK3 kinase activity, e.g. N-benzyl-3,4-dihydroxy-benzylidene-cyanoacetamide α-cyano-(3,4-dihydroxy)-]N-benzylcinnamamide (Tyrphostin AG 490), prodigiosin 25-C(PNU156804), [4-(4′-hydroxyphenyl)-amino-6,7-dimethoxyquinazoline] (WHI-P131), [4-(3′-bromo-4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g. mono-citrate (also called CP-690,550), or a compound as disclosed in WO2004052359 or WO2005066156;
mediators, e.g. agonists or modulators of S1P receptor activity, e.g. FTY720 optionally phosphorylated or an analog thereof, e.g. 2-amino-2-[4-(3-benzyloxyphenylthio)-2-chlorophenyl]ethyl-1,3-propanediol optionally phosphorylated or 1-{4-[1-(4-cyclohexyl-3-trifluoromethyl-benzyloxyimino)-ethyl]-2-ethyl-benzyl}-azetidine-3-carboxylic acid or its pharmaceutically acceptable salts;
immunosuppressive monoclonal antibodies, e.g., monoclonal antibodies to leukocyte receptors, e.g., Blys/BAFF receptor, MHC, CD2, CD3, CD4, CD7, CD8, CD20, e.g. rituximab (Rituxan®, ibritumomab tiuxetan conjugated to ¹¹¹In or ⁹⁰Y (Zevalin®), ¹³¹I tositumumab ( )Bexxar®), CD25, CD28, CD33, e.g. gemtuzumab (Mylotarg®, CD40, CD45, CD52, e.g. Alemtuzumab (Campath-I®), CD58, CD80, CD86, IL-2 receptor, e.g. dacluzimab, IL6 receptor (e.g. tocilizumab), IL-12 receptor, IL-17 receptor, IL-23 receptor or their ligands;
other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g.
   LEA29Y; or an anti-CTLA4 agent, such as ipilimumab:
mediators, e.g. inhibitors of adhesion molecule activities, e.g. LFA-1 antagonists, ICAM-1 or -3 antagonists, VCAM-4 antagonists or VLA-4 antagonists,
mediators, e.g. antagonists of CCR9 activity,
mediators, e.g. inhibitors, of MIF activity,
5-aminosalicylate (5-ASA) agents, such as sulfasalazine, Azulfidine®, Asacol®, Dipentum®, Pentasa®, Rowasa®, Canasa®, Colazal®, e.g. drugs containing mesalamine; e.g mesalazine in combination with heparin;
mediators, e.g. inhibitors, of TNF-alpha activity, e.g. including antibodies which bind to TNF-alpha, e.g. infliximab (Remicade®), thalidomide, lenalidomide, golimumab, adalimumab (Humira®, fully human immunoglobulin G (IgG1) monoclonal antibody that is specific for human TNF alpha), etanercept (Enbrel®), certolizumab pegol (Cimzia®, CDP 870),
nitric oxide releasing non-steriodal anti-inflammatory drugs (NSAIDs), e.g. including COX-inhibiting NO-donating drugs (CINOD);
phosphordiesterase, e.g. mediators, such as inhibitors of PDE4B activity,
mediators, e.g. inhibitors, of caspase activity,
mediators, e.g. agonists, of the G protein coupled receptor GPBAR1,
mediators, e.g. inhibitors, of ceramide kinase activity,
‘multi-functional anti-inflammatory’ drugs (MFAIDs), e.g. cytosolic phospholipase A2 (cPLA2) inhibitors, such as membrane-anchored phospholipase A2 inhibitors linked to glycosaminoglycans;
antibiotics, such as penicillins, cephalosporins, erythromycins, tetracyclines, sulfonamides, such as sulfadiazine, sulfisoxazole; sulfones, such as dapsone; pleuromutilins, fluoroquinolones, e.g. metronidazole, quinolones such as ciprofloxacin; levofloxacin; probiotics and commensal bacteria e.g. Lactobacillus, Lactobacillus reuteri;
antiviral drugs, such as ribivirin, vidarabine, acyclovir, ganciclovir, zanamivir, oseltamivir phosphate, famciclovir, atazanavir, amantadine, didanosine, efavirenz, foscarnet, indinavir, lamivudine, nelfinavir, ritonavir, saquinavir, stavudine, valacyclovir, valganciclovir, civacir, zidovudine,
mediators, e.g. inhibitors of the blood protein “complement 5a”, such as pexelizumab,
serum phosphorus controlling agents, e.g. sevelamer carbonate (Renagel®), phosphate binders that reduces high serum phosphate levels in renal disease patients, such as lanthanum carbonate (Fosrenol®).
mediators, e.g. agonists, of GPBAR1 mediator activity, e.g. including antibodies and low molecular weight compounds;
ceramide kinase inhhibitors other than the compounds of the present invention.

Anti-inflammatory drugs which are prone to be useful in combination with a compound of the present invention include e.g. non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); cyclooxygenase-2 (COX-2) inhibitors such as celecoxib; inhibitors of phosphodiesterase type IV (PDE-IV); antagonists of the chemokine receptors, especially CCR1, CCR2, and CCR3; cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrate, fenofibrate and benzafibrate), and probucol; anticholinergic agents such as muscarinic antagonists (ipratropium bromide); other compounds such as theophylline, sulfasalazine and aminosalicylates, e.g. 5-aminosalicylic acid and prodrugs thereof, antirheumatics.

Antiallergic drugs which are prone to be useful in combination with a compound of the present invention include e.g. antihistamines (H1-histamine antagonists), e.g. bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and non-steroidal anti-asthmatics such as β2-agonists (terbutaline, metaproterenol, fenoterol, isoetharine, albuterol, bitolterol, salmeterol and pirbuterol), theophylline, cromolyn sodium, atropine, ipratropium bromide, leukotriene antagonists (zafirlukast, montelukast, praniukast, iralukast, pobilukast, SKB-106,203), leukotriene biosynthesis inhibitors (zileuton, BAY-1005); bronchodilators, antiasthmatics (mast cell stabilizers).

Anticancer drugs which are prone to be useful as a combination partner with an mTOR inhibitor, e.g. prone to be useful according to the present invention, e.g. include
i. a steroid; e.g. prednisone.
ii. an adenosine-kinase-inhibitor; which targets, decreases or inhibits nucleobase, nucleoside, nucleotide and nucleic acid metabolisms, such as 5-Iodotubercidin, which is also known as 7H-pyrrolo[2,3-d]pyrimidin-4-amine, 5-iodo-7-β-D-ribofuranosyl.
iii. an adjuvant; which enhances the 5-FU-TS bond as well as a compound which targets, decreases or inhibits, alkaline phosphatase, such as leucovorin, levamisole.
iv. an adrenal cortex antagonist; which targets, decreases or inhibits the activity of the adrenal cortex and changes the peripheral metabolism of corticosteroids, resulting in a decrease in 17-hydroxycorticosteroids, such as mitotane.
v. an AKT pathway inhibitor; such as a compound which targets, decreases or inhibits Akt, also known as protein kinase B (PKB), such as deguelin, which is also known as 3H-bis[1]benzopyrano[3,4-b:6′,5′-e]pyran-7(7aH)-one, 13,13a-dihydro-9,10-dimethoxy-3,3-dimethyl-, (7aS,13aS); and triciribine, which is also known as 1,4,5,6,8-pentaazaacenaphthylen-3-amine, 1,5-dihydro-5-methyl-1-β-D-ribofuranosyl; KP372-1 (QLT394).
vi. an alkylating agent; which causes alkylation of DNA and results in breaks in the DNA molecules as well as cross-linking of the twin strands, thus interfering with DNA replication and transcription of RNA, such as chlorambucil, chlormethine, cyclophosphamide, ifosfamide, melphalan, estramustine; nitrosueras, such as carmustine, fotemustine, lomustine, streptozocin (streptozotocin, STZ), BCNU; Gliadel; dacarbazine, mechlorethamine, e.g. in the form of a hydrochloride, procarbazine, e.g. in the form of a hydrochloride, thiotepa, temozolomide, nitrogen mustard, mitomycin, altretamine, busulfan, estramustine, uramustine. Cyclophosphamide can be administered, e.g., in the form as it is marketed, e.g., under the trademark CYCLOSTIN®; ifosfamide as HOLOXAN®, temozolomide as TEMODAR®, nitrogen mustard as MUSTARGEN®, estramustine as EMYCT®, streptozocin as ZANOSAR®).
vii. an angiogenesis inhibitor; which targets, decreases or inhibits the production of new blood vessels, e.g. which targets methionine aminopeptidase-2 (MetAP-2), macrophage inflammatory protein-1 (MIP-1alpha), CCL5, TGF-beta, lipoxygenase, cyclooxygenase, and topoisomerase, or which indirectly targets p21, p53, CDK2 and collagen synthesis, e.g. including fumagillin, which is known as 2,4,6,8-decatetraenedioic acid, mono[(3R,4S,5S,6R)-5-methoxy-4-[(2R,3R)-2-methyl-3-(3-methyl-2-butenyl)oxiranyl]-1-oxaspiro[2.5]oct-6-yl]ester, (2E,4E,6E,8E)-(9CI); shikonin, which is also known as 1,4-naphthalenedione, 5,8-dihydroxy-2-[(1R)-1-hydroxy-4-methyl-3-pentenyl]-(9CI); tranilast, which is also known as benzoic acid, 2-[[3-(3,4-dimethoxyphenyl)-1-oxo-2-propenyl]amino]; ursolic acid; suramin; bengamide or a derivative thereof, thalidomide, TNP-470.
viii. an anti-androgen; which blocks the action of androgens of adrenal and testicular origin which stimulate the growth of normal and malignant prostatic tissue, such as nilutamide; bicalutamide (CASODEX®), which can be formulated, e.g., as disclosed in U.S. Pat. No. 4,636,505.
ix. an anti-estrogen; which antagonizes the effect of estrogens at the estrogen receptor level, e.g. including an aromatase inhibitor, which inhibits the estrogen production, i.e. the conversion of the substrates androstenedione and testosterone to estrone and estradiol, respectively, e.g. including atamestane, exemestane, formestane, aminoglutethimide, roglethimide, pyridoglutethimide, trilostane, testolactone, ketokonazole, vorozole, fadrozole, anastrozole, letrozole, toremifene; bicalutamide; flutamide; tamoxifen, tamoxifen citrate; tamoxifen; fulvestrant; raloxifene, raloxifene hydrochloride. Tamoxifen may be e.g. administered in the form as it is marketed, e.g., NOLVADEX®; and raloxifene hydrochloride is marketed as EVISTA®. Fulvestrant may be formulated as disclosed in U.S. Pat. No. 4,659,516 and is marketed as FASLODEX®.
x. an anti-hypercalcemia agent; which is used to treat hypercalcemia, such as gallium (III) nitrate hydrate; and pamidronate disodium.
xi. an antimetabolite; which inhibits or disrupts the synthesis of DNA resulting in cell death, such as folic acids, e.g. methotrexate, pemetrexed, raltitrexed; purins, e.g. 6-mercaptopurine, cladribine, clofarabine; fludarabine, thioguanine (tioguanine), 6-thioguanine, nelarabine (compound 506), tiazofurin (inhibits inosine monophosphate dehydrogenase and guanosine triphosphate pools), pentostatin (deoxycoformycin); cytarabine; flexuridine; fluorouracil; 5-fluorouracil (5-FU), floxuridine (5-FUdR), capecitabine; gemcitabine; gemcitabine hydrochloride; hydroxyurea (e.g. Hydrea®); DNA de-methylating agents, such as 5-azacytidine (Vidaza®) and decitabine; fluoromethylene deoxycitidine (FmdC), 5-aza-2′-deoxycytidine, troxacitabine (L-isomer cytosine analogue), edatrexate; Capecitabine and gemcitabine can be administered e.g. in the marketed form, such as XELODA® and GEMZAR®.
xii. an apoptosis inducer; which induces the normal series of events in a cell that leads to its death, e.g. selectively inducing the X-linked mammalian inhibitor of apoptosis protein XIAP, or e.g. downregulating BCL-xL; such as ethanol, 2-[[3-(2,3-dichlorophenoxy)propyl]amino]; gambogic acid; embelin, which is also known as 2,5-cyclohexadiene-1,4-dione, 2,5-dihydroxy-3-undecyl-(9CI); arsenic trioxide arsenic trioxide (TRISENOX®).
xiii. an aurora kinase inhibitor; which targets, decreases or inhibits later stages of the cell cycle from the G2/M check point all the way through to the mitotic checkpoint and late mitosis; such as binucleine 2, which is also known as methanimidamide, N′-[1-(3-chloro-4-fluorophenyl)-4-cyano-1H-pyrazol-5-yl]-N,N-dimethyl-(9CI).
xiv. a Bruton's Tyrosine Kinase (BTK) inhibitor; which targets, decreases or inhibits human and murine B cell development; such as terreic acid.
xv. a calcineurin inhibitor; which targets, decreases or inhibits the T cell activation pathway, such as cypermethrin, which is also known as cyclopropanecarboxylic acid, 3-(2,2-dichloroethenyl)-2,2-dimethyl-,cyano(3-phenoxyphenyl)methyl ester; deltamethrin, which is also known as cyclopropanecarboxylic aci, 3-(2,2-dibromoethenyl)-2,2-dimethyl-(S)-cyano(3-phenoxyphenyl)methyl ester, (1R,3R); fenvalerate, which is also known as benzeneacetic acid, 4-chloro-α-(1-methylethyl)-,cyano(3-phenoxyphenyl)methyl ester; and Tyrphostin 8; but excluding cyclosporin or FK506.
xvi. a CaM kinase II inhibitor; which targets, decreases or inhibits CaM kinases; constituting a family of structurally related enzymes that include phosphorylase kinase, myosin light chain kinase, and CaM kinases I-IV; such as 5-isoquinolinesulfonic acid, 4-[(2S)-2-[(5-isoquinolinylsulfonyl)methylamino]-3-oxo-3-(4-phenyl-1-piperazinyl)propyl]phenyl ester (9CI); benzenesulfonamide, N-[2-[[[3-(4-chlorophenyl)-2-propenyl]methyl]amino]methyl]phenyl]-N-(2-hydroxyethyl)-4-methoxy.
xvii. a CD45 tyrosine phosphatase inhibitor; which targets, decreases or inhibits dephosphorylating regulatory pTyr residues on Src-family protein-tyrosine kinases, which aids in the treatment of a variety of inflammatory and immune disorders; such as phosphonic acid, [[2-(4-bromophenoxy)-5-nitrophenyl]hydroxymethyl].
xviii. a CDC25 phosphatase inhibitor; which targets, decreases or inhibits overexpressed dephosphorylate cyclin-dependent kinases in tumors; such as 1,4-naphthalenedione, 2,3-bis[(2-hydroyethyl)thio].
xix. a CHK kinase inhibitor; which targets, decreases or inhibits overexpression of the antiapoptotic protein Bcl-2; such as debromohymenialdisine. Targets of a CHK kinase inhibitor are CHK1 and/or CHK2.
xx. a controlling agent for regulating genistein, olomucine and/or tyrphostins; such as daidzein, which is also known as 4H-1-benzopyran-4-one, 7-hydroxy-3-(4-hydroxyphenyl); Iso-Olomoucine, and Tyrphostin 1.
xxi. a cyclooxygenase inhibitor; e.g. including Cox-2 inhibitors; which targets, decreases or inhibits the enzyme Cox-2 (cyclooxygenase-2); such as 1H-indole-3-acetamide, 1-(4-chlorobenzoyl)-5-methoxy-2-methyl-N-(2-phenylethyl); 5-alkyl substituted 2-arylaminophenylacetic acid and derivatives, e.g. celecoxib (CELEBREX®), rofecoxib (VIOXX®), etoricoxib, valdecoxib; or a 5-alkyl-2-arylaminophenylacetic acid, e.g., 5-methyl-2-(2′-chloro-6′-fluoroanilino)phenyl acetic acid, lumiracoxib; and celecoxib.
xxii. a cRAF kinase inhibitor; which targets, decreases or inhibits the up-regulation of E-selectin and vascular adhesion molecule-1 induced by TNF; such as 3-(3,5-dibromo-4-hydroxybenzylidene)-5-iodo-1,3-dihydroindol-2-one; and benzamide, 3-(dimethylamino)-N-[3-[(4-hydroxybenzoyl)amino]-4-methylphenyl]. Raf kinases play an important role as extracellular signal-regulating kinases in cell differentiation, proliferation, and apoptosis. A target of a cRAF kinase inhibitor includes, but is not limited, to RAF1.
xxiii. a cyclin dependent kinase inhibitor; which targets, decreases or inhibits cyclin dependent kinase playing a role in the regulation of the mammalian cell cycle; such as N9-isopropyl-olomoucine; olomoucine; purvalanol B, which is also known as Benzoic acid, 2-chloro-4-[[2-[[(1R)-1-(hydroxymethyl)-2-methylpropyl]amino]-9-(1-methylethyl)-9H-purin-6-yl]amino]-(9CI); roascovitine; indirubin, which is also known as 2H-indol-2-one, 3-(1,3-dihydro-3-oxo-2H-indol-2-ylidene)-1,3-dihydro-(9CI); kenpaullone, which is also known as indolo[3,2-d][1]benzazepin-6(5H)-one, 9-bromo-7,12-dihydro-(9CI); purvalanol A, which is also known as 1-Butanol, 2-[[6-[(3-chlorophenyl)amino]-9-(1-methylethyl)-9H-purin-2-yl]amino]-3-methyl-, (2R)-(9CI); indirubin-3′-monooxime. Cell cycle progression is regulated by a series of sequential events that include the activation and subsequent inactivation of cyclin dependent kinases (Cdks) and cyclins. Cdks are a group of serine/threonine kinases that form active heterodimeric complexes by binding to their regulatory subunits, cyclins. Examples of targets of a cyclin dependent kinase inhibitor include, but are not limited to, CDK, AHR, CDK1, CDK2, CDK5, CDK4/6, GSK3beta, and ERK.
xxiv. a cysteine protease inhibitor; which targets, decreases or inhibits cystein protease which plays a vital role in mammalian cellular turnover and apotosis; such as 4-morpholinecarboxamide, N-[(1S)-3-fluoro-2-oxo-1-(2-phenylethyl)propyl]amino]-2-oxo-1-(phenylmethyl)ethyl].
xxv. a DNA intercalator; which binds to DNA and inhibits DNA, RNA, and protein synthesis; such as plicamycin, dactinomycin.
xxvi. a DNA strand breaker; which causes DNA strand scission and results in inhibition of DNA synthesis, ininhibition of RNA and protein synthesis; such as bleomycin.
xxvii. an E3 Ligase inhibitor; which targets, decreases or inhibits the E3 ligase which inhibits the transfer of ubiquitin chains to proteins, marking them for degradation in the proteasome; such as N-((3,3,3-trifluoro-2-trifluoromethyl)propionyl)sulfanilamide.
xxviii. an endocrine hormone; which by acting mainly on the pituitary gland causes the suppression of hormones in males, the net effect being a reduction of testosterone to castration levels; in females, both ovarian estrogen and androgen synthesis being inhibited; such as leuprolide; megestrol, megestrol acetate.
xxix. compounds targeting, decreasing or inhibiting the activity of the epidermal growth factor family of receptor tyrosine kinases (EGFR, ErbB2, (HER-2), ErbB3, ErbB4 as homo- or heterodimers), such as compounds, proteins or antibodies which inhibit members of the EGF receptor tyrosine kinase family, e.g. EGF receptor, ErbB1, ErbB2, ErbB3 and ErbB4 or bind to EGF or EGF-related ligands, and are in particular those compounds, proteins or monoclonal antibodies generically and specifically disclosed in WO 9702266, e.g. the compound of ex. 39, EP0564409, WO9903854, EP0520722, EP0566226, EP0787722, EP0837063, U.S. Pat. No. 5,747,498, WO9810767, WO9730034, WO9749688, WO9738983 and, especially, WO9630347, e.g. a compound known as CP 358774, WO9633980, e.g. a compound known as ZD 1839; and WO 9503283, e.g. a compound known as ZM105180, e.g including the dual acting tyrosine kinase inhibitor (ErbB1 and ErbB2) lapatinib (GSK572016), e.g. lapatinib ditosylate; panituzumab, trastuzumab (HERCEPTIN®), cetuximab, iressa, OSI-774, CI-1033, EKB-569, GW-2016, E1.1, E2.4, E2.5, E6.2, E6.4, E2.11, E6.3 or E7.6.3, 7H-pyrrolo-[2,3-d]pyrimidine derivatives which are e.g. disclosed in WO03013541, erlotinib, gefitinib. Erlotinib can be administered in the form as it is marketed, e.g. TARCEVA®, and gefitinib as IRESSA®, human monoclonal antibodies against the epidermal growth factor receptor including ABX-EGFR.
xxx. an EGFR, PDGFR tyrosine kinase inhibitor; such as EGFR kinase inhibitors including tyrphostin 23, tyrphostin 25, tyrphostin 47, tyrphostin 51 and tyrphostin AG 825; 2-propenamide, 2-cyano-3-(3,4-dihydroxyphenyl)-N-phenyl-(2E); tyrphostin Ag 1478; lavendustin A; 3-pyridineacetonitrile, α-[(3,5-dichlorophenyl)methylene]-, (αZ); an example of an EGFR, PDGFR tyrosine kinase inhibitor e.g. includes tyrphostin 46. PDGFR tyrosine kinase inhibitor including tyrphostin 46. Targets of an EGFR kinase inhibitor include guanylyl cyclase (GC-C) HER2, EGFR, PTK and tubulin.
xxxi. a farnesyltransferase inhibitor; which targets, decreases or inhibits the Ras protein; such as a-hydroxyfarnesylphosphonic acid; butanoic acid, 2-[[(2S)-2-[[(2S,3S)-2-[[(2R)-2-amino-3-mercaptopropyl]amino]-3-methylpentyl]oxy]-1-oxo-3-phenylpropyl]amino]-4-(methylsulfonyl)-,1-methylethyl ester, (2S); manumycin A; L-744,832 or DK8G557, tipifarnib (R115777), SCH66336 (lonafarnib), BMS-214662,
xxxii. a Flk-1 kinase inhibitor; which targets, decreases or inhibits Flk-1 tyrosine kinase activity; such as 2-propenamide, 2-cyano-3-[4-hydroxy-3,5-bis(1-methylethyl)phenyl]-N-(3-phenylpropyl)-(2E). A target of a Flk-1 kinase inhibitor includes, but is not limited to, KDR.
xxxiii. a Glycogen synthase kinase-3 (GSK3) inhibitor; which targets, decreases or inhibits glycogen synthase kinase-3 (GSK3); such as indirubin-3′-monooxime. Glycogen Synthase Kinase-3 (GSK-3; tau protein kinase 1), a highly conserved, ubiquitously expressed serine/threonine protein kinase, is involved in the signal transduction cascades of multiple cellular processes. which is a protein kinase that has been shown to be involved in the regulation of a diverse array of cellular functions, including protein synthesis, cell proliferation, cell differentiation, microtubule assembly/disassembly, and apoptosis.
xxxiv. a histone deacetylase (HDAC) inhibitor; which inhibits the histone deacetylase and which possess anti-proliferative activity; such as compounds disclosed in WO0222577, especially N-hydroxy-3-[4-[[(2-hydroxyethyl)[2-(1H-indol-3-yl)ethyl]-amino]methyl]phenyl]-2E-2-propenamide, and N-hydroxy-3-[4-[[[2-(2-methyl-1H-indol-3-yl)-ethyl]-amino]methyl]phenyl]-2E-2-propenamide and pharmaceutically acceptable salts thereof; suberoylanilide hydroxamic acid (SAHA); [4-(2-amino-phenylcarbamoyl)-benzyl]-carbamic acid pyridine-3-ylmethyl ester and derivatives thereof; butyric acid, pyroxamide, trichostatin A, oxamflatin, apicidin, depsipeptide; depudecin; trapoxin, HC toxin, which is also known as cyclo[L-alanyl-D-alanyl-(□S,2S)-□-amino-□-oxooxiraneoctanoyl-D-prolyl] (9CI); sodium phenylbutyrate, suberoyl bis-hydroxamic acid; Trichostatin A, BMS-27275, pyroxamide, FR-901228, valproic acid.
xxxv. a HSP90 inhibitor; which targets, decreases or inhibits the intrinsic ATPase activity of HSP90; degrades, targets, decreases or inhibits the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, e.g., 17-allylamino, 17-demethoxygeldanamycin (17AAG), a geldanamycin derivative; other geldanamycin-related compounds; radicicol and HDAC inhibitors. Other examples of an HSP90 inhibitor include geldanamycin, 17-demethoxy-17-(2-propenylamino). Potential indirect targets of an HSP90 inhibitor include FLT3, BCR-ABL, CHK1, CYP3A5*3 and/or NQ01*2. Nilotinib is an example of an BCR-ABL tyrosine kinase inhibitor.
xxxvi.a I-kappa B-alpha kinase inhibitor (IKK); which targets, decreases or inhibits NF-kappaB, such as 2-propenenitrile, 3-[(4-methylphenyl)sulfonyl]-(2E).
xxxvii. an insulin receptor tyrosine kinase inhibitor; which modulates the activities of phosphatidylinositol 3-kinase, microtubule-associated protein, and S6 kinases; such as hydroxyl-2-naphthalenylmethylphosphonic acid, LY294002.
xxxviii. a c-Jun N-terminal kinase (JNK) kinase inhibitor; which targets, decreases or inhibits Jun N-terminal kinase; such as pyrazoleanthrone and/or epigallocatechin gallate. Jun N-terminal kinase (JNK), a serine-directed protein kinase, is involved in the phosphorylation and activation of c-Jun and ATF2 and plays a significant role in metabolism, growth, cell differentiation, and apoptosis. A target for a JNK kinase inhibitor includes, but is not limited to, DNMT.
xxxix a microtubule binding agent; which acts by disrupting the microtubular network that is essential for mitotic and interphase cellular function; such as vinca alkaloids, e.g. vinblastine, vinblastine sulfate; vincristine, vincristine sulfate; vindesine; vinorelbine; taxanes, such as taxanes, e.g. docetaxel; paclitaxel; discodermolides; colchicine, epothilones and derivatives thereof, e.g. epothilone B or a derivative thereof. Paclitaxel is marketed as TAXOL®; docetaxel as TAXOTERE®; vinblastine sulfate as VINBLASTIN R.P®; and vincristine sulfate as FARMISTIN®. Also included are the generic forms of paclitaxel as well as various dosage forms of paclitaxel. Generic forms of paclitaxel include, but are not limited to, betaxolol hydrochloride. Various dosage forms of paclitaxel include, but are not limited to albumin nanoparticle paclitaxel marketed as ABRAXANE®; ONXOL®, CYTOTAX®. Discodermolide can be obtained, e.g., as disclosed in U.S. Pat. No. 5,010,099. Also included are Epotholine derivatives which are disclosed in U.S. Pat. No. 6,194,181, WO98/0121, WO9825929, WO9808849, WO9943653, WO9822461 and WO0031247. Especially preferred are Epotholine A and/or B.
xl. a mitogen-activated protein (MAP) kinase-inhibitor; which targets, decreases or inhibits Mitogen-activated protein, such as benzenesulfonamide, N-[2-[[[3-(4-chlorophenyl)-2-propenyl]methyl]amino]methyl]phenyl]-N-(2-hydroxyethyl)-4-methoxy. The mitogen-activated protein (MAP) kinases are a group of protein serine/threonine kinases that are activated in response to a variety of extracellular stimuli and mediate signal transduction from the cell surface to the nucleus. They regulate several physiological and pathological cellular phenomena, including inflammation, apoptotic cell death, oncogenic transformation, tumor cell invasion, and metastasis.
xli. a MDM2 inhibitor; which targets, decreases or inhibits the interaction of MDM2 and the p53 tumor suppressor; such as trans-4-iodo, 4′-boranyl-chalcone.
xlii. a MEK inhibitor; which targets, decreases or inhibits the kinase activity of MAP kinase MEK; such as sorafenib, e.g. Nexavar® (sorafenib tosylate), butanedinitrile, bis[amino[2-aminophenyl)thio]methylene]. A target of a MEK inhibitor includes, but is not limited to ERK. An indirect target of a MEK inhibitor includes, but is not limited to, cyclin D1.
xliii: a matrix metalloproteinase inhibitor (MMP) inhibitor; which targets, decreases or inhibits a class of protease enzyme that selectively catalyze the hydrolysis of polypeptide bonds including the enzymes MMP-2 and MMP-9 that are involved in promoting the loss of tissue structure around tumors and facilitating tumor growth, angiogenesis, and metastasissuch as actinonin, which is also known as butanediamide, N-4-hydroxy-N1-[(1S)-1-[[(2S)-2-(hydroxymethyl)-1-pyrrolidinyl]carbonyl]-2-methylpropyl]-2-pentyl-, (2R)-(9CI); epigallocatechin gallate; collagen peptidomimetic and non-peptidomimetic inhibitors; tetracycline derivatives, e.g., hydroxamate peptidomimetic inhibitor batimastat; and its orally-bioavailable analogue marimastat, prinomastat, metastat, neovastat, tanomastat, TAA211, BMS-279251, BAY 12-9566, MMI270B or AAJ996. A target of a MMP inhibitor includes, but is not limited to, polypeptide deformylase.
xliv. a NGFR tyrosine-kinase-inhibitor; which targets, decreases or inhibits nerve growth factor dependent p140^{c-trk} tyrosine phosphorylation; such as tyrphostin AG 879. Targets of a NGFR tyrosine-kinase-inhibitor include, but are not limited to, HER2, FLK1, FAK, TrkA, and/or TrkC. An indirect target inhibits expression of RAF1.
xlv. a p38 MAP kinase inhibitor, including a SAPK2/p38 kinase inhibitor; which targets, decreases or inhibits p38-MAPK, which is a MAPK family member, such as phenol, 4-[4-(4-fluorophenyl)-5-(4-pyridinyl)-1H-imidazol-2-yl]. An example of a a SAPK2/p38 kinase inhibitor includes, but is not limited to, benzamide, 3-(dimethylamino)-N-[3-[(4-hydroxybenzoyl)amino]-4-methylphenyl]. A MAPK family member is a serine/threonine kinase activated by phosphorylation of tyrosine and threonine residues. This kinase is phosphorylated and activated by many cellular stresses and inflammatory stimuli, thought to be involved in the regulation of important cellular responses such as apoptosis and inflammatory reactions.
xlvi. a p56 tyrosine kinase inhibitor; which targets, decreases or inhibits p56 tyrosine kinase, which is an enzyme that is a lymphoid-specific src family tyrosine kinase critical for T-cell development and activation; such as damnacanthal, which is also known as 2-anthracenecarboxaldehyde,9,10-dihydro-3-hydroxy-1 methoxy-9,10-dioxo, Tyrphostin 46. A target of a p56 tyrosine kinase inhibitor includes, but is not limited to, Lck. Lck is associated with the cytoplasmic domains of CD4, CD8 and the beta-chain of the IL-2 receptor, and is thought to be involved in the earliest steps of TCR-mediated T-cell activation.
xlvii. a PDGFR tyrosine kinase inhibitor; targeting, decreasing or inhibiting the activity of the C-kit receptor tyrosine kinases (part of the PDGFR family), such as targeting, decreasing or inhibiting the activity of the c-Kit receptor tyrosine kinase family, especially inhibiting the c-Kit receptor. Examples of targets of a PDGFR tyrosine kinase inhibitor includes, but are not limited to PDGFR, FLT3 and/or c-KIT; such as tyrphostin AG 1296; tyrphostin 9; 1,3-butadiene-1,1,3-tricarbonitrile,2-amino-4-(1H-indol-5-yl); N-phenyl-2-pyrimidine-amine derivative, e.g. imatinib, IRESSA®. PDGF plays a central role in regulating cell proliferation, chemotaxis, and survival in normal cells as well as in various disease states such as cancer, atherosclerosis, and fibrotic disease. The PDGF family is composed of dimeric isoforms (PDGF-AA, PDGF-BB, PDGF-AB, PDGF-CC, and PDGF-DD), which exert their cellular effects by differentially binding to two receptor tyrosine kinases. PDGFR-α and PDGFR-β have molecular masses of −170 and 180 kDa, respectively.
xlviii. a phosphatidylinositol 3-kinase inhibitor; which targets, decreases or inhibits PI 3-kinase; such as wortmannin, which is also known as 3H-Furo[4,3,2-de]indeno[4,5-h]-2-benzopyran-3,6,9-trione, 11-(acetyloxy)-1,6b,7,8,9a,10,11,11b-octahydro-1-(methoxymethyl)-9a,11b-dimethyl-, (1S,6bR,9aS,11R,11bR)— (9CI); 8-phenyl-2-(morpholin-4-yl)-chromen-4-one; quercetin, quercetin dihydrate. PI 3-kinase activity has been shown to increase in response to a number of hormonal and growth factor stimuli, including insulin, platelet-derived growth factor, insulin-like growth factor, epidermal growth factor, colony-stimulating factor, and hepatocyte growth factor, and has been implicated in processes related to cellular growth and transformation. An example of a target of a phosphatidylinositol 3-kinase inhibitor includes, but is not limited to, Pi3K.
xlix. a phosphatase inhibitor; which targets, decreases or inhibits phosphatase; such as cantharidic acid; cantharidin; and L-leucinamide, N-[4-(2-carboxyethenyl)benzoyl]glycyl-L-α-glutamyl-(E). Phosphatases remove the phosphoryl group and restore the protein to its original dephosphorylated state. Hence, the phosphorylation-dephosphorylation cycle can be regarded as a molecular “on-off” switch.
l. a platinum agent; which contains platinum and inhibit DNA synthesis by forming interstrand and intrastrand cross-linking of DNA molecules; such as carboplatin; cisplatin; oxaliplatin; cisplatinum; satraplatin and platinum agents such as ZD0473, BBR3464. Carboplatin can be administered, e.g., in the form as it is marketed, e.g. CARBOPLAT®; and oxaliplatin as ELOXATIN®.
li. a protein phosphatase inhibitor, including a PP1 and PP2 inhibitor and a tyrosine phosphatase inhibitor; which targets, decreases or inhibits protein phosphatase. Examples of a PP1 and PP2A inhibitor include cantharidic acid and/or cantharidin. Examples of a tyrosine phosphatase inhibitor include, but are not limited to, L-P-bromotetramisole oxalate; 2(5H)-furanone,4-hydroxy-5-(hydroxymethyl)-3-(1-oxohexadecyl)-, (5R); and benzylphosphonic acid.
   The term “a PP1 or PP2 inhibitor”, as used herein, relates to a compound which targets, decreases or inhibits Ser/Thr protein phosphatases. Type I phosphatases, which include PP1, can be inhibited by two heat-stable proteins known as Inhibitor-1 (I-1) and Inhibitor-2 (I-2). They preferentially dephosphorylate a subunit of phosphorylase kinase. Type II phosphatases are subdivided into spontaneously active (PP2A), CA²⁺-dependent (PP2B), and Mg²⁺-dependent (PP2C) classes of phosphatases. The term “tyrosine phosphatase inhibitor”, as used here, relates to a compounds which targets, decreases or inhibits tyrosine phosphatase. Protein tyrosine phosphatases (PTPs) are relatively recent additions to the phosphatase family. They remove phosphate groups from phosphorylated tyrosine residues of proteins. PTPs display diverse structural features and play important roles in the regulation of cell proliferation, differentiation, cell adhesion and motility, and cytoskeletal function. Examples of targets of a tyrosine phosphatase inhibitor include, but are not limited to, alkaline phosphatase (ALP), heparanase, PTPase, and/or prostatic acid phosphatase.
lii. a PKC inhibitor and a PKC delta kinase inhibitor: The term “a PKC inhibitor”, as used herein, relates to a compound which targets, decreases or inhibits protein kinase C as well as its isozymes. Protein kinase C (PKC), a ubiquitous, phospholipid-dependent enzyme, is involved in signal transduction associated with cell proliferation, differentiation, and apoptosis. Examples of a target of a PKC inhibitor include, but are not limited to, MAPK and/or NF-kappaB. Examples of a PKC inhibitor include, but are not limited to, 1-H-pyrrolo-2,5-dione,3-[1-[3-(dimethylamino)propyl]-1H-indol-3-yl]-4-(1H-indol-3-yl); bisindolylmaleimide IX; sphingosine, which is known as 4-octadecene-1,3-diol, 2-amino-, (2S,3R,4E)-(9CI); staurosporine, which is known as 9,13-Epoxy-1H,9H-diindolo[1,2,3-gh:3′,2′,1′-Im]pyrrolo[3,4-j][1,7]benzodiazonin-1-one, staurosporine derivatives such as disclosed in EP0296110, e.g. midostaurin; 2,3,10,11,12,13-hexahydro-10-methoxy-9-methyl-11-(methylamino)-, (9S,10R,11R,13R)-(9CI); tyrphostin 51; and hypericin, which is also known as phenanthro[1,10,9,8-opqra]perylene-7,14-dione, 1,3,4,6,8,13-hexahydroxy-10,11-dimethyl-, stereoisomer (6C₁₋₇C1,8CI,9CI), UCN-01, safingol, BAY 43-9006, bryostatin 1, perifosine; llmofosine; RO 318220 and RO 320432; GO 6976; Isis 3521; LY333531/LY379196. The term “a PKC delta kinase inhibitor”, as used herein, relates to a compound which targets, decreases or inhibits the delta isozymes of PKC. The delta isozyme is a conventional PKC isozymes and is Ca²⁺-dependent. An example of a PKC delta kinase inhibitor includes, but is not limited to, Rottlerin, which is also known as 2-Propen-1-one, 1-[6-[(3-acetyl-2,4,6-trihydroxy-5-methylphenyl)methyl]-5,7-dihydroxy-2,2-dimethyl-2H-1-benzopyran-8-yl]-3-phenyl-, (2E)-(9CI).
liii. a polyamine synthesis inhibitor; which targets, decreases or inhibits polyamines spermidine; such as DMFO, which is also known as (−)-2-difluoromethylornithin; N1, N12-diethylspermine 4HCl. The polyamines spermidine and spermine are of vital importance for cell proliferation, although their precise mechanism of action is unclear. Tumor cells have an altered polyamine homeostasis reflected by increased activity of biosynthetic enzymes and elevated polyamine pools.
liv. a proteosome inhibitor; which targets, decreases or inhibits proteasome, such as aclacinomycin A; gliotoxin; PS-341; MLN 341; bortezomib; velcade. Examples of targets of a proteosome inhibitor include, but are not limited to, O(2)(−)-generating NADPH oxidase, NF-kappaB, and/or farnesyltransferase, geranyltransferase I.
lv. a PTP1B inhibitor; which targets, decreases or inhibits PTP1B, a protein tyrosine kinase inhibitor; such as L-leucinamide, N-[4-(2-carboxyethenyl)benzoyl]glycyl-L-α-glutamyl-,(E).
lvi. a protein tyrosine kinase inhibitor including a SRC family tyrosine kinase inhibitor; a Syk tyrosine kinase inhibitor; and a JAK-2 and/or JAK-3 tyrosine kinase inhibitor; The term “a protein tyrosine kinase inhibitor”, as used herein, relates to a compound which targets, decreases or inhibits protein tyrosine kinases. Protein tyrosine kinases (PTKs) play a key role in the regulation of cell proliferation, differentiation, metabolism, migration, and survival. They are classified as receptor PTKs and non-receptor PTKs. Receptor PTKs contain a single polypeptide chain with a transmembrane segment. The extracellular end of this segment contains a high affinity ligand-binding domain, while the cytoplasmic end comprises the catalytic core and the regulatory sequences. Examples of targets of a tyrosine kinase inhibitor include, but are not limited to, ERK1, ERK2, Bruton's tyrosine kinase (Btk), JAK2, ERK ½, PDGFR, and/or FLT3. Examples of indirect targets include, but are not limited to, TNFalpha, NO, PGE2, IRAK, iNOS, ICAM-1, and/or E-selectin. Examples of a tyrosine kinase inhibitor include, but are not limited to, tyrphostin AG 126; tyrphostin Ag 1288; tyrphostin Ag 1295; geldanamycin; and genistein.
Non-receptor tyrosine kinases include members of the Src, Tec, JAK, Fes, AbI, FAK, Csk, and Syk families. They are located in the cytoplasm as well as in the nucleus. They exhibit distinct kinase regulation, substrate phosphorylation, and function. Deregulation of these kinases has also been linked to several human diseases.
The term “a SRC family tyrosine kinase inhibitor”, as used herein, relates to a compound which targets, decreases or inhibits SRC. Examples of a SRC family tyrosine kinase inhibitor include, but are not limited to, PP1, which is also known as 1H-pyrazolo[3,4-d]pyrimidin-4-amine, 1-(1,1-dimethylethyl)-3-(1-naphthalenyl)-(9CI); and PP2, which is also known as 1H-Pyrazolo[3,4-d]pyrimidin-4-amine, 3-(4-chlorophenyl)-1-(1,1-dimethylethyl)-(9CI).
The term “a Syk tyrosine kinase inhibitor”, as used herein, relates to a compound which targets, decreases or inhibits Syk. Examples of targets for a Syk tyrosine kinase inhibitor include, but are not limited to, Syk, STAT3, and/or STAT5. An example of a Syk tyrosine kinase inhibitor includes, but is not limited to, piceatannol, which is also known as 1,2-benzenediol, 4-[(1E)-2-(3,5-dihydroxyphenyl)ethenyl]-(9CI).
The term “a Janus (JAK-2 and/or JAK-3) tyrosine kinase inhibitor”, as used herein, relates to a compound which targets, decreases or inhibits janus tyrosine kinase. Janus tyrosine kinase inhibitor are shown anti-leukemic agents with anti-thrombotic, anti-allergic and immunosuppressive properties. Targets of a JAK-2 and/or JAK-3 tyrosine kinase inhibitor include, but are not limited to, JAK2, JAK3, STAT3. An indirect target of an JAK-2 and/or JAK-3 tyrosine kinase inhibitor includes, but is not limited to CDK2. Examples of a JAK-2 and/or JAK-3 tyrosine kinase inhibitor include, but are not limited to, Tyrphostin AG 490; and 2-naphthyl vinyl ketone.
Compounds which target, decrease or inhibit the activity of c-Abl family members and their gene fusion products, e.g. include PD180970; AG957; or NSC 680410.
lvii. a retinoid; which target, decrease or inhibit retinoid dependent receptors; such as isotretinoin, tretinoin, alitretinoin, bexarotene, e.g. including an agent which interact with retinoic acid responsive elements on DNA, such as isotretinoin (13-cis-retinoic acid).
lviii. a RNA polymerase II elongation inhibitor; which targets, decreases or inhibits insulin-stimulated nuclear and cytosolic p70S6 kinase in CHO cells; targets, decreases or inhibits RNA polymerase II transcription, which may be dependent on casein kinase II; and targets, decreases or inhibits germinal vesicle breakdown in bovine oocytes; such as 5,6-dichloro-1-beta-D-ribofuranosylbenzimidazole.
lvix. a serine/threonine kinase inhibitor; which inhibits serine/threonine kinases; such as 2-aminopurine. An example of a target of a serine/threonine kinase inhibitor includes, but is not limited to, dsRNA-dependent protein kinase (PKR). Examples of indirect targets of a serine/threonine kinase inhibitor include, but are not limited to, MCP-1, NF-kappaB, elF2alpha, COX2, RANTES, IL8, CYP2A5, IGF-1, CYP2B1, CYP2B2, CYP2H1, ALAS-1, HIF-1, erythropoietin, and/or CYP1A1.
lx. a sterol biosynthesis inhibitor; which inhibits the biosynthesis of sterols such as cholesterol; such as terbinadine. Examples of targets for a sterol biosynthesis inhibitor include, but are not limited to, squalene epoxidase, and CYP2D6.
lxi. a topoisomerase inhibitor; including a topoisomerase I inhibitor and a topoisomerase II inhibitor. Examples of a topoisomerase I inhibitor include, but are not limited to, topotecan, gimatecan, irinotecan, camptothecan and its analogues, 9-nitrocamptothecin and the macromolecular camptothecin conjugate PNU-166148 (compound A1 in WO9917804); 10-hydroxycamptothecin e.g. the acetate salt; idarubicin, e.g. the hydrochloride; irinotecan, e.g. the hydrochloride; etoposide; teniposide; topotecan, topotecan hydrochloride; doxorubicin; epirubicin, epirubicin hydrochloride; 4′-epidoxorubicin, mitoxantrone, mitoxantrone, e.g. the hydrochloride; daunorubicin, daunorubicin hydrochloride, valrubicin, dasatinib (BMS-354825). Irinotecan can be administered, e.g., in the form as it is marketed, e.g., under the trademark CAMPTOSAR®. Topotecan can be administered, e.g., in the form as it is marketed, e.g., under the trademark HYCAMTIN®. The term “topoisomerase II inhibitor”, as used herein, includes, but is not limited to, the anthracyclines, such as doxorubicin, including liposomal formulation, e.g., CAELYX®, daunorubicin, including liposomal formulation, e.g., DAUNOSOME®, epirubicin, idarubicin and nemorubicin; the anthraquinones mitoxantrone and losoxantrone; and the podophillotoxines etoposide and teniposide. Etoposide is marketed as ETOPOPHOS®; teniposide as VM 26-BRISTOL®; doxorubicin as ADRIBLASTIN® or ADRIAMYCIN®; epirubicin as FARMORUBICIN® idarubicin as ZAVEDOS®; and mitoxantrone as NOVANTRON®).
lxii. VEGFR tyrosine kinase inhibitor; which targets, decreases and/or inhibits the known angiogenic growth factors and cytokines implicated in the modulation of normal and pathological angiogenesis. The VEGF family (VEGF-A, VEGF-B, VEGF-C, VEGF-D) and their corresponding receptor tyrosine kinases [VEGFR-1 (FIt-1), VEGFR-2 (Flk-1, KDR), and VEGFR-3 (Flt-4)] play a paramount and indispensable role in regulating the multiple facets of the angiogenic and lymphangiogenic processes. An example of a VEGFR tyrosine kinase inhibitor includes 3-(4-dimethylaminobenzylidenyl)-2-indolinone. Compounds which target, decrease or inhibit the activity of VEGFR are especially compounds, proteins or antibodies which inhibit the VEGF receptor tyrosine kinase, inhibit a VEGF receptor or bind to VEGF, and are in particular those compounds, proteins or monoclonal antibodies generically and specifically disclosed in WO9835958, e.g. 1-(4-chloroanilino)-4-(4-pyridylmethyl) phthalazine or a pharmaceutical acceptable salt thereof, e.g. the succinate, or in WO0009495, WO0027820, WO0059509, WO9811223, WO0027819 and EP0769947; e.g. those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et al in Cancer Res. 58, 1998, 3209-3214, and by J. Mordenti et al in Toxicologic Pathology, Vol. 27, no. 1, pp 14-21, 1999; in WO0037502 and WO9410202; Angiostatin, described by M. S. O'Reilly et al, Cell 79, 1994, 315-328; Endostatin described by M. S. O'Reilly et al, Cell 88, 1997, 277-285; anthranilic acid amides; ZD4190; ZD6474 (vandetanib); SU5416; SU6668, AZD2171 (Recentin®); or anti-VEGF antibodies or anti-VEGF receptor antibodies, e.g. RhuMab (bevacizumab). By antibody is meant intact monoclonal antibodies, polyclonal antibodies, multispecific antibodies formed from at least 2 intact antibodies, and antibodies fragments so long as they exhibit the desired biological activity. an example of an VEGF-R2 inhibitor e.g. includes axitinib,
lxiii. a gonadorelin agonist, such as abarelix, goserelin, goserelin acetate,
lxiv. a compound which induce cell differentiation processes, such as retinoic acid, alpha-, gamma- or 8-tocopherol or alpha-, gamma- or 8-tocotrienol.
lxv. a bisphosphonate, e.g. including etridonic, clodronic, tiludronic, pamidronic, alendronic, ibandronic, risedronic and zoledronic acid.
lxvi. a heparanase inhibitor which prevents heparan sulphate degradation, e.g. PI-88,
lxvii. a biological response modifier, preferably alymphokine or interferons, e.g. interferon alpha,
lxviii. a telomerase inhibitor, e.g. telomestatin,
lxix. mediators, such as inhibitors of catechol-O-methyltransferase, e.g. entacapone,
lxx: ispinesib, permetrexed (Alimta®), sunitinib (SU11248), diethylstilbestrol (DES), BMS224818 (LEA29Y), vatanalib,
lxxi somatostatin or a somatostatin analogue, such as octreotide (Sandostatin® or Sandostatin LAR®).
lxxii. Growth Hormone-Receptor Antagonists, such as pegvisomant, filgrastim or pegfilgrastim, or interferon alpha:
lxxiii. monoclonal antibodies, e.g. useful for leukemia (AML) treatment, such as alemtuzumab (Campath®), rituximab/Rituxan®), gemtuzumab, (ozogamicin, Mylotarg®), epratuzumab.
lxxiv. altretamine, amsacrine, asparaginase (Elspar®), denileukin diftitox, masoprocol, pegaspargase, gemtuzumab (MYLOTARG®),
lxxv. a phosphodiesterase inhibitor, e.g. anagrelide (Agrylin®), Xagrid®).
lxxvi. a cancer vaccine, such as MDX-1379.
lxxvii. an immunosuppressive monoclonal antibody, e.g., monoclonal antibodies to leukocyte receptors,
e.g. CD20, such as rituximab (Rituxan®, ibritumomab tiuxetan conjugated to ¹¹¹In or ⁹⁰Y (Zevalin®), ¹³¹I tositumumab ( )Bexxar®),
CD33, such as gemtuzumab (Mylotarg®,
CD52, e.g. alemtuzumab (Campath-I®),
or their ligands;

Anesthetics drugs which are prone to be useful in combination with a compound of the present invention include e.g. include ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocalne, phenazopyridine.

In the following Examples all temperatures are in degrees Celsius (° C.).

The following abbreviations are used:

### DMF N,N-dimethylformamide

EDC (1-ethyl-3-[3-dimethylaminopropyl]carbodiimide
ETOAc ethyl acetate
HOAt 1-hydroxy-7-azabenzotriazole,
rt room temperature
THF tetrahydrofurane
TLC: thin layer chromatography

### EXAMPLE 1

### Adamantane-1-carboxylic Acid (2-benzoylamino-benzothiazol-6-yl)-amide

100 mg of N-(6-amino-benzothiazol-2-yl)-benzamide, 250 mg of adamantane carboxylic acid, 24.5 mg of HOAt, 247 μl of triethylamine and 126 μl of EDC/free base) are dissolved in 2 ml of dry DMF and stirred at 60° for 2 hours. The mixture obtained is diluted with EtOAc, and extracted with 1N HCl and 5% aqueous NaHCO₃ solution. From the organic layer obtained solvent is evaporated and the evaporation residue is subjected to chromatography. Adamantane-1-carboxylic acid (2-benzoylamino-benzothiazol-6-yl)-amide is obtained.

### EXAMPLE 2

### Adamantane-1-carboxylic Acid (2-acetylamino-benzothiazol-6-yl)-amide

A mixture of 50 mg of adamantane-1-carboxylic acid (2-amino-benzothiazol-6-yl)-amide and a catalytic amount of 4-dimethylaminopyridine in 5 ml THF and 70 μl of acetic anhydride are stirred overnight at 50°. The mixture obtained is diluted with EtOAc and washed with 0.1 N HCl and 5% aqueous NaHCO₃. Solvent is evaporated and the evaporation residue is subjected to chromatography. Adamantane-1-carboxylic acid (2-acetylamino-benzothiazol-6-yl)-amide is obtained.

Analogously to the methods as described in Examples 1 or 2, but using appropriate starting materials (intermediates) compounds of formula

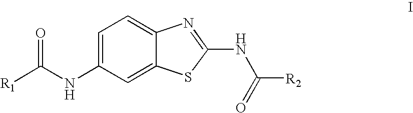

wherein R₁ and R₂ are defined in TABLE 1 below, showing analyticyl data from mass spectroscopy (MS) and/or having a melting (Fp) as set out in the column headed “MS or Fp” in TABLE 1 are obtained;

TABLE 1

| EX | R₁ | R₂ | MS, Fp (° C.), R_{f} |
|---|---|---|---|
| 1 | 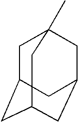 | 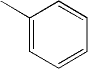 | Fp 246-248 MH+ 432.4 R_{f} = 0.58 in toluene:i-propanol = 4:1 |
| 2 | 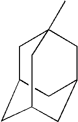 | CH₃ | MNa+ 392.1 R_{f} = 0.82 in CH₂Cl₂:EtOAc = 1:1 |
| 3 | 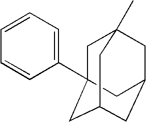 | 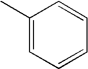 | Fp 257-265 MNa+ 530.2 R_{f} = 0.65 in toluene:i-propanol = 4:1 |
| 4 | CH₃—(CH₂)₁₁—CH₂— | 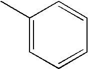 | Fp 160-167 MH+ 480.6 R_{f} = 0.63 in toluene:i-propanol 4:1 |
| 5 | 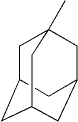 | 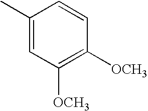 | MNa+ 514.1 R_{f} = 0.70 iin toluene:i-propanol 4:1 |
| 6 | 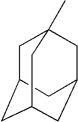 | 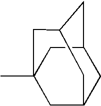 | MNa+ 512 R_{f} = 0.36 in CH₂Cl₂:EtOAc 1:1 |

“EX” in TABLE 1 designates the compound number. The R_{f} values in TABLE 1 are determined in TLC on silicagel in the solvent mixture as indicated.

### Preparation of Intermediates (Starting Materials)

### EXAMPLE A

### N-(6-Amino-benzothiazol-2-yl)-benzamide

Aa. N-(6-nitro-benzothiazol-2-yl)-benzamide)

1 g of 2-amino-6-nitro-benzothiazole, 1.04 ml triethylamine and 0.87 ml of benzoylchloride and a catalytic amount of 4-dimethylaminopyridine is dissolved in 50 ml of THF and heated to reflux for 4 hours. The primarily formed 3-acyl product isomerizes to the desired 2-amide during heating. Excess of benzoylchloride is hydrolyzed with water overnight at rt. The mixture obtained is diluted with ETOAc and N-(6-nitro-benzothiazol-2-yl)-benzamide) precipitates and is filtrated off.

Ab. N-(6-amino-benzothiazol-2-yl)-benzamide)

300 mg of N-(6-nitro-benzothiazol-2-yl)-benzamide) in 50 ml of acetic acid are treated with 1 g of powdered tin with stirring for 6 hours at rt. The reaction mixture obtained is neutralized with aqueous NaOH and the mixture obtained is extracted with CH₂Cl₂. Solvent is evaporated and N-(6-amino-benzothiazol-2-yl)-benzamide is obtained.

### EXAMPLE B

### Adamantane-1-carboxylic Acid (2-amino-benzothiazol-6-yl)-amide

Ba) Benzothiazole-2,6-diamine

2 g of 2-amino-6-nitro-benzothiazole and 3 g of Raney-nickel in 200 ml of CH₃OH in methanol/THF are treated with hydrogen at rt. The mixture obtained is filtrated and from the filtration residue obtained solvent is evaporated. Benzothiazole-2,6-diamine is obtained.

Bbl Adamantane-1-carboxylic Acid (2-amino-benzothiazol-6-yl)-amide

A mixture of benzothiazole-2,6-diamine as obtained in step Ba) is 1.1 g of adamantane carboxylic acid, 1.3 ml of EDC (free base), 83 mg of HOAt and 1.2 ml of diisopropylethylamine in 30 ml of DMF is stirred for 3 hours at 40° The mixture obtained is diluted with EtOAc and washed with 0.1 N HCl and 5% aqueous NaHCO₃ solution. From the mixture obtained solvent is evaporated. Adamantane-1-carboxylic acid (2-amino-benzothiazol-6-yl)-amide is obtained.

1. A compound of formula 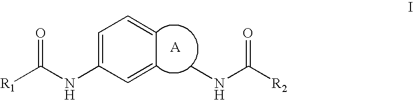
wherein
R₁ is a straight chain, branched or cyclic aliphatic, aromatic or heterocyclyl group comprising at least 8 carbon atoms, e.g. 8 to 22,
R₂ is a straight chain, branched or cyclic aliphatic, aromatic or heterocyclic group comprising from 1 to 12 carbon atoms, and
ring A is heterocycyl, fused with the phenyl ring to which ring A is attached comprising 5 or 6 ring members, and 1 to 4 heteroatoms selected from N, S, O; with the proviso that the compounds
N-[2-(acetylamino)-6-benzothiazolyl]-tricyclo[3.3.1.13,7]decane-1-carboxamide,
N-[2-(benzoylamino)-6-benzothiazolyl]-tricyclo[3.3.1.13,7]decane-1-carboxamide,
N-[2-(benzoylamino)-6-benzothiazolyl]-3-chloro-benzo[b]thiophene-2-carboxamide,
2,3-Dihydro-N-[2-[(1-oxobutyl)amino]-6-benzothiazolyl]-1,4-benzodioxin-6-carboxamide,
N-[2-(acetylamino)-6-benzothiazolyl]-3-chloro-benzo[b]thiophene-2-carboxamide,
N-[2-(butyrylamino)-1,3-benzothiazol-6-yl]-3-chloro-1-benzothiophene-2-carboxamide,
N-[2-(butyrylamino)-1,3-benzothiazol-6-yl]-1-benzofuran-2-carboxamide,
N-[2-[(cyclohexylcarbonyl)-amino]-6-benzothiazolyl]-tricyclo[3.3.1.13,7]decane-1-carboxamide,
adamantane-1-carboxylic acid [2-(adamantan-1-yl)-carbonylamino-benzothiazol-6-yl]-amide,
N-[2,3-dihydro-3-oxo-6-[(1-oxohexadecyl)-amino]-benzo[b]-thien-2-yl]-5-nitro-1H-indazole-1-carboxamide, and
N-[2,3-dihydro-3-oxo-6-[(1-oxohexadecyl)-amino]-benzo[b]-thien-2-yl]-2,3,5,6-tetrafluoro-4-mercapto benzamide
are excluded.

2. 2,6-Diamido-benzothiazoles or 2,6-diamido-benzoxazoles, wherein the carbonyl group of the aminocarbonyl group in position 6 is substituted by a straight chain, branched or cyclic aliphatic, aromatic or heterocyclyl group comprising at least 8 carbon atoms, and the carbonyl group of the aminocarbonyl group in position 2 is substituted by a straight chain, branched or cyclic aliphatic, aromatic or heterocyclic group comprising from 1 to 8 carbon, with the proviso of claim 1.

3. A compound according to claim 1, which is a compound of formula 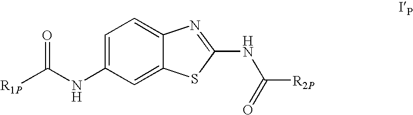
wherein
R_{1P} is (C₈₋₂₂)alkyl, or (C₈₋₁₈)cycloalkyl optionally substituted by phenyl; and
R_{2P} is (C₁₋₈)alkyl, (C₃₋₁₂)cycloalkyl, or phenyl, e.g. including (C₁₋₄)alkoxyphenyl, (C₁₋₄)dialkoxyphenyl,
with the proviso that the compounds
N-[2-(acetylamino)-6-benzothiazolyl]-tricyclo[3.3.1.13,7]decane-1-carboxamide,
N-[2-(benzoylamino)-6-benzothiazolyl]-tricyclo[3.3.1.13,7]decane-1-carboxamide, and
adamantane-1-carboxylic acid [2-(adamantan-1-yl)-carbonylamino-benzothiazol-6-yl]-amide
are excluded.

4. A compound as claimed in claim 1 selected from the group
3-Phenyl-adamantane-1-carboxylic acid (2-benzoylamino-benzothiazol-6-yl)-amide,
N-(6-Tetradecanoylamino-benzothiazol-2-yl)-benzamide, and
Adamantane-1-carboxylic acid [2-(3,4-dimethoxy-benzoylamino)-benzothiazol-6-yl]-amide.

5. A compound of claim 1 in the form of a salt.

6. A compound of claim 1 for use as a pharmaceutical.

7. A pharmaceutical composition comprising a compound of claim 1 in association with at least one pharmaceutical excipient.

8. A method of treating disorders mediated by ceramide kinase activity, which treatment comprises administering to a subject in need of such treatment an effective amount of a compound of claim 1.

9. A compound of claim 1 for the manufacture of a medicament for the treatment of disorders which are mediated by ceramide kinase activity.

10. A combination of a compound of claim 1, with at least one second drug substance.

11. A compound of claim 1 in combination with at least one second drug substance for use as a pharmaceutical, pharmaceutical excipient or for the treatment of disorders which are mediated by cermide kinase activity.
